(19) United States
(12) Patent Application Publication
(10) Pub. No.: US 2026/0209693 A1
(43) Pub. Date: Jul. 23, 2026

(54) CULTURE METHODS

(71) Applicant: CELLULAREVOLUTION LIMITED, Newcastle upon Tyne (GB)

(72) Inventors: Martina MIOTTO, Whitley Bay (GB); Che John CONNON, North Shields (GB); Azzeldin MADKOUR, Newcastle upon Tyne (GB)

(21) Appl. No.: 19/140,191

(22) PCT Filed: Dec. 19, 2023
(86) PCT No.: PCT/GB2023/053308

§ 371 Date: Jun. 17, 2025

(30) Foreign Application Priority Data
Dec. 20, 2022 (GB) 2219240.5

(51) Int. Cl. C12N 5/00

(52) U.S. Cl. CPC C12N 5/0062; C12N 2501/734; C12N 2513/00; C12N 2531/00

(57) ABSTRACT

The present invention relates to a method for reducing aggregation in a cell cluster-based cell culture system, such as a microcarrier-based or a spheroid-based cell culture system.

The present invention relates to a method for reducing aggregation in a cell cluster-based cell culture system, such as a microcarrier-based or a spheroid-based cell culture system.

### BACKGROUND

It is well-known that regenerative medicine, and in particular cell-therapy techniques, has been expanding its repertoire in terms of both purposeful cell types and applications, raising the potential to cure a wide range of diseases. However, the high number of cells required for some treatments (this can be up to 1 billion) presents, in terms of cell manufacture, a major challenge.

Furthermore, as the final product is often represented by the cells themselves, numerous requirements have to be met under the current regulatory framework for cellular therapy products.

Thus, implementing and adapting industrial processes, previously developed for large-scale mammalian cell culture for the production of biologics, has been attempted in order to sustain such a great demand for cells and to meet the required conditions.

While substantial increases in cell yield have been achieved, some other critical production steps are still undergoing optimization. For instance, improved cell harvesting methods for easier, more effective, and less invasive recovery of the expanded cells from their culture substrates are required.

In order to scale up culture systems for the production of cell or cell-based therapies for industrial processes, it is often advantageous to facilitate growth of cells in cell clusters (such as on microcarriers, or as spheroids) in a bioreactor. A spheroid culture system provides a similar physicochemical environment in vivo by facilitating cell-cell and cell-matrix interaction to overcome the limitations of traditional monolayer cell culture. Microcarriers are particles suspended in a cell culture medium, which serve to effectively multiply the available surface area upon which the cells may adhere and grow. Microcarriers may typically be spherical particles upon which several hundred cells may grow. By providing such a large surface area for cell proliferation, microbeads provide an environment in which cells can settle so that adherent cells, such as stem cells or progenitor cells, can grow even in a suspended culture environment.

However, aggregation or clumping of cell clusters (such as aggregation or clumping of spheroids or cell-populated microcarriers) in a cell culture can reduce access of cells to nutrients and can result in a reduction of growth.

It is an object of the present invention to provide methods that reduce aggregation or clumping of cell clusters (such as spheroids or cell-populated microcarriers), and thereby ameliorate some of the problems associated with the prior art.

### BRIEF SUMMARY OF THE DISCLOSURE

In one aspect of the present invention, there is provided a method of reducing aggregation in a cell cluster-based cell culture system, the method comprising the step of:
(i) culturing a cell population comprising two or more cell clusters in a cell culture medium for a culture period, wherein the cell culture medium comprises an exogenous protease, wherein the exogenous protease is an exogenous metalloprotease and/or cysteine protease.

In one aspect of the present invention, there is provided a method of reducing aggregation in a cell cluster-based cell culture system, the method comprising the step of:
(i) culturing a cell population comprising two or more cell clusters in a cell culture medium for a culture period, wherein the cell culture medium comprises a supplemented protease, wherein the protease is a metalloprotease and/or cysteine protease.

In another aspect of the invention, there is provided a cell culture vessel comprising a cell culture medium, two or more cell clusters and an exogenous protease, wherein the exogenous protease is an exogenous metalloprotease and/or cysteine protease, optionally wherein the two or more cell clusters comprise a cell population in the log phase of growth.

In another aspect of the invention, there is provided a cell culture vessel comprising a cell culture medium, two or more cell clusters and a supplemented protease, wherein the protease is a metalloprotease and/or cysteine protease, optionally wherein the two or more cell clusters comprise a cell population in the log phase of growth.

Suitably, in a method and/or cell culture vessel of the invention, the two or more cell clusters may each comprise a microcarrier. Such cell clusters are also referred to as cell-populated or cell-loaded microcarriers herein. Accordingly, suitably, the two or more cell clusters may each be cell-populated microcarriers.

Suitably, in a method and/or cell culture vessel of the invention, the two or more cell clusters may each be a spheroid, organoid or tissue.

Suitably, in the method of the invention, aggregation may be cell-mediated aggregation.

Suitably, in a method of the invention, the culturing step may comprise all or part of the log phase of growth of the cell population.

Suitably, in a method of the invention, the culture period may be at least 24 hours.

Suitably, in a method of the invention, the cell culture system may be a continuous cell culture system or a batch cell culture system, optionally wherein the system comprises a bioreactor.

Suitably, in a cell culture vessel of the invention, the vessel may be part of a continuous cell culture system or a batch cell culture system, optionally wherein the system comprises a bioreactor.

Suitably, in a method and/or cell culture vessel of the invention, the metalloprotease may be collagenase and/or dispase.

Suitably, in a method and/or cell culture vessel of the invention, the metalloprotease may be collagenase.

Suitably, in a method and/or cell culture vessel of the invention, the collagenase may be a collagenase selected from the group consisting of: collagenase type I, collagenase type II, collagenase type III, collagenase type IV, collagenase type V, collagenase type VI, and collagenase type VII. Notably, collagenase type I to type VII are enzyme compositions comprising collagenase at increasing levels of purity, with collagenase type VII being pure collagenase.

Suitably, in a method and/or cell culture vessel of the invention, the collagenase may be collagenase type I or collagenase type VII. These are the collagenases that are used herein to exemplify the invention, although the invention is not limited thereto.

Suitably, in a method and/or cell culture vessel of the invention, the metalloprotease may be a dispase.

Suitably, in a method and/or cell culture vessel of the invention, the dispase may be dispase I or dispase II.

Suitably, in a method and/or cell culture vessel of the invention, the cysteine protease may be of the CA clan.

Suitably, the cysteine protease of the CA clan may be selected from the group consisting of ficin, papain, bromelain, cathepsin K and calpain. More suitably, the cysteine protease of the CA clan may be ficin and/or papain.

Suitably, in a method and/or cell culture vessel of the invention, the two or more cell clusters may each comprise a microcarrier, and the microcarrier may be a bead.

Suitably, in a method and/or cell culture vessel of the invention, the two or more cell clusters may each comprise a microcarrier and the microcarrier (such as a bead) may comprise a material selected from the group consisting of: plastic, polymer, glass, and metal.

Suitably, in a method and/or cell culture vessel of the invention, the cell population may comprise cells selected from the group consisting of: skin, muscle, cervical, breast, and prostate cells.

Suitably, in a method of the invention, the cell culture medium may be supplemented with the protease (such as collagenase and/or dispase) one or more times during the step of culturing.

Suitably, a method of the invention may comprise culturing the cell population in a cell culture medium lacking the metalloprotease (such as collagenase and/or dispase) and/or cysteine protease (such as ficin and/or papain) before and/or after step (i).

Suitably, in a method and/or cell culture vessel of the invention, the metalloprotease (such as collagenase and/or dispase) cysteine protease (such as ficin and/or papain) may be present in the cell culture medium at a concentration which maintains the cell culture at about 50% to 100% confluency.

Suitably, in a method of the invention, the culturing step may not comprise a step of washing the cells (e.g. it some examples it does not comprise the step of washing an artificial support (such as a microcarrier) on which the cells are grown, or a step of washing the cell cluster itself (e.g. it does not comprise the step of washing a spheroid, organoid or tissue, or washing a cell-populated microcarrier).

Suitably, in the method of the invention, the metalloprotease (such as collagenase and/or dispase) and/or cysteine protease (such as ficin and/or papain) may be present in the cell culture medium at a concentration which allows for continuous detachment of a portion of the cells from the cell clusters.

Suitably, in a method of the invention, the metalloprotease (for example collagenase and/or dispase) and/or cysteine protease (such as ficin and/or papain) may be present in the cell culture medium at a concentration which allows for the ratio of the rate of detachment of cells (e.g. detachment from the cell cluster e.g. from the spheroid or the microcarrier) to the rate of growth of the cells to be in the range of 0.3:1 to 1:3.

Suitably, in a method of the invention, the detached cells, or a proportion of the detached cells, may be harvested at least once during the culturing step.

Suitably, in a method of the invention, the detached cells, or a proportion of the detached cells, may be harvested more than once during the culturing step.

Suitably, a method of the invention may comprise adjusting the metalloprotease (for example collagenase and/or dispase) and/or cysteine protease (such as ficin and/or papain) concentration and/or frequency of application to adjust the rate of cell detachment, wherein increasing the concentration and/or frequency of application increases the rate of continuous cell detachment and decreasing the concentration and/or frequency of application decreases the rate of continuous cell detachment.

Suitably, in a method of the invention, the cell culture system may comprise a bioreactor. Suitably, in a method and/or cell culture vessel of the invention, the cell culture system may comprise a population of cell clusters (e.g. a population of (cell-populated) microcarriers, a population of spheroids, a population of organoids, or a population of tissues).

### BRIEF DESCRIPTION OF THE DRAWINGS

Embodiments of the invention are further described hereinafter with reference to the accompanying drawings, in which:

FIG. 1 shows that collagenase can successfully detach C2C12 cells in bulk similar to TrypLE (exemplified using collagenase type I). Graphs showing similar number of detached cells (A) and viability (B) using TrypLE and collagenase without altering cell adhesion (C) proliferation (D) and differentiation (E) potential (Scale bar 200 μm).

FIG. 2 shows that collagenase is capable of detaching cells in a dose-dependent manner without altering the cell phenotype (exemplified using collagenase type I). Images showing attached (A) and detached (B) cells in the presence of varying concentrations of collagenase. The amounts of collagenase (U/mL) are shown in the Figure. The equivalent amounts in mg/mL for collagenase type I are: 2.635 U/mL (0.012 mg/mL); 3.500 U/mL (0.016 mg/mL); and 4.375 U/mL (0.020 mg/mL). (C) Graph showing increased cell detachment with increasing concentration of collagenase. (D) Representative images showing only expression of undifferentiated cell marker (Pax7) and not of differentiated cell marker (MHC) in attached cells at the end of the experiment (Scale bar 200 μm).

FIG. 3 is a schematic showing the supplementation of collagenase as a strategy for continuous detachment of adherent cells in two systems: on flat and on microcarrier surfaces and in two conditions (static and dynamic). Exemplified using collagenase type I.

FIG. 4 shows the continuous detachment in a flat-static system. Cell detachment using 1.75 U/mL (equivalent to 0.008 mg/mL) of collagenase (collagenase type I) shows similar number of attached (A) and detached (B) cells over 3 weeks. Quantification showing similar number of attached (C) and detached (D) cells over time suggesting steady state of cell proliferation and cell yield (Scale bar 200 μm).

FIG. 5 shows the continuous detachment in a flat-dynamic system. Exemplified using collagenase type I. (A) Images showing similar number of attached cells over a week in a flat dynamic system. (B) Quantification showing similar number of cells were harvest on day 7 compared to seeded cells on day 0. (C&D) Representative images showing expression of undifferentiated cell marker, Pax7 in both detached (C) and attached (D) cells (Scale bar 200 μm).

FIG. 6 shows the continuous cell detachment from microcarrier surfaces when supplementing the media with collagenase (exemplified using collagenase type I). Stained cell nuclei showing microcarrier aggregation in controls (B) caused by cell overgrowth, while collagenase addition inhibits microcarrier bridging and cellular overgrowth (C). Quantification showing (D) average number of weekly detached cells using collagenase in the static system (over 27 days) and (E) total number of harvested cells from the microcarriers at the end of the experiment (Scale bars 200 μm and 1000 μm).

FIG. 7 shows attached cell behaviour. Cells detached from microcarriers using collagenase (A) can re-attach on flat tissue culture treated surface in serum plus media as well as the cells recovered from the microcarriers at the end of the experiment (B). (C) The collagenase-detached cells are also capable to attach on fresh microcarriers and stay in the continuous process in serum free media with collagenase. Exemplified using collagenase type I (Scale bars 200 μm).

FIG. 8 shows continuous detachment in a microcarrier-dynamic system. Quantification showing daily (A) and weekly (B) number of cells detached using collagenase over 27 days in the dynamic system. (C) Representative images of nuclei stained cell on microcarriers and (D) final quantification showing seeded and harvested number of cells at the end of the experiment. Exemplified using collagenase type I.

FIG. 9 shows attached cell behaviour. Cells detached from microcarriers using collagenase (detached) can re-attach on flat tissue culture treated surface in serum plus media as well as the cells recovered from the microcarriers at the end of the experiment (attached). (C) Collagenase-detached cells can be cryopreserved and are capable to re-attach and grow on tissue culture treated cell culture plastic once defrosted (defrosted cryopreserved). Exemplified using collagenase type I (Scale bars 200 μm).

FIG. 10 shows that collagenase can successfully detach fish skin fibroblasts in bulk. After 20 h incubation with collagenase the cells are detached (A) and after collection (B) they are capable of reattaching (5 h after seeding, C) and proliferating (48 h after seeding, D). Exemplified using collagenase type I used at a concentration of 1505 U/mL (which is equivalent to 7 mg/mL, Scale bars 500 μm).

FIG. 11 shows that dispase can successfully detach C2C12 cells continuously. Confluence of growing cells is maintained during the 3 days (attached, A) whilst cells are being detached (detached, B) at a comparable rate over time. Further experiments have also shown the same effect over 21 days (data not shown). Exemplified using dispase I used at a concentration of 0.0008 U/mL (which is equivalent to 0.00006 mg/mL or 60 ng/ml, Scale bars 200 μm).

FIG. 12 shows that collagenase can successfully detach C2C12 cells continuously. Confluence of growing cells is maintained during the 3 days (attached) whilst cells are being detached (detached) at a comparable rate over time. Exemplified using collagenase type VII. (A) Collagenase VII used at a concentration of 106.35 Units/mL (equivalent to 0.07 mg/mL); (B) Collagenase VII used at a concentration of 53.18 Units/mL (equivalent to 0.035 mg/mL, Scale bars 200 μm).

FIG. 13 shows Ficin and Performase® detach cells in a dose dependency manner. Images showing C2C12 cells growing on surfaces 6 days after incubation with different concentrations of Ficin (A) and Performase® (B) added to serum-free media (Scale bars 250 μm).

FIG. 14 shows continuous cell detachment with Ficin (A) or Performase® (B). Images taken at day 0 and 10 showing the initial confluency (after cell seeding) and the confluency after continuous enzyme exposure, respectively. Images at day 10 show the confluency on the growth surface (attached) and the cells that detached during the last 3 days of the experiment (detached, Scale bars 250 μm).

FIG. 15 shows Bulk detachment with Ficin or PerformaseR. C2C12 cells can be detached in bulk with Ficin (A) and Performase® (B) once added to the culture media. Cells detached already after 30 minutes with complete detachment occurring after 150 minutes (Scale bars 250 μm).

FIG. 16 shows cell viability after Ficin or Performase® exposure. C2C12 cells detached in bulk with Ficin or Performase® maintained a high cell viability after 4 h of exposure to high enzyme concentrations.

### DEFINITIONS

As used herein, “about” will be understood by persons of ordinary skill, and will vary to some extent depending on the context in which it is used. If there are uses of the term which are not clear to persons of ordinary skill given the context in which it is used, “about” will mean up to plus or minus 10% of the particular value.

The terms “comprise”, “comprises” and “comprising” as used herein, specify the presence of the stated features, integers, steps, operations, elements, and/or components of an aspect or embodiment of the invention, but do not preclude the presence or addition of one or more other features, integers, steps, operations, elements, components, and/or groups thereof.

The phrase “consisting essentially of” means that the scope of an aspect or embodiment is to be interpreted to encompass the specified materials or steps recited in the aspect or embodiment, including any materials or steps which do not materially affect the invention defined by the aspect or embodiment. Thus, the term “consisting essentially of” when used in a claim of this invention is not intended to be interpreted to be equivalent to “comprising.”

The term “cell cluster” refers to a group (e.g. a ball or a layer etc) of cells. It refers to a plurality of adjoining or interconnected cells. A cell cluster may be formed from e.g. at least adjoining cells (wherein each cell is in direct contact (in other words touching) with at least one other cell within the cluster). For example, the cluster may comprise at least 10, at least 10², at least 10³, at least 10⁴, at least 10⁵, at least 10⁶, at least 10⁷, at least 10⁸, or at least 10⁹ etc adjoining cells. In a preferred example, the adjoining cells are interconnected.

As used herein, “adjoining” refers to cells that are connected to each other in a manner that forms a ball, layer etc of cells. The adjoining cells retain the cluster form (e.g. ball, layer etc) when placed in a solution such as cell culture medium. Adjoining cells may be in direct contact e.g, wherein they adhere to or touch each other in a manner that forms a cluster of cells. Alternatively, adjoining cells may be connected indirectly in a manner that forms a cluster of cells, such as by virtue of the presence of a matrix, support, or scaffold (e.g. an extracellular matrix), wherein the matrix, support, or scaffold connects the adjoining cells into the cluster. The term “cell cluster” may also be referred to as a cell aggregate herein. Examples of cell clusters include a cell layer (e.g. on a microcarrier), a spheroid, an organoid, a tissue, or any combination thereof.

As would be clear to a person of skill in the art, cells may form a cell cluster, with or without an artificial support. In one example, cells may form a cell cluster in the absence of an artificial support (e.g. the cell cluster may be a spheroid, an organoid or a tissue, where the cells grow and/or attach to each other, in other words, the cells self-aggregate to form a cell cluster). In another example, cells may be adjoining cells (i.e. a cell cluster) by virtue of growing on the same artificial support (e.g. cells growing on a microcarrier, where the cells grow and/or attach to the support (as well as optionally growing and/or attaching to each other)). The term “cell cluster” herein encompasses all of these examples, and thus encompasses cells/cell layers that are attached to an artificial support such as a microcarrier (e.g. a cell-populated microcarrier), as well as spheroids, organoids or tissues (e.g. micro-tissues).

Spheroids are spherical cellular units that are generally cultured as free-floating aggregates and are of low complexity in mirroring tumour organization. Spheroids are well-designed cell aggregates that can be used as engineered cancer models. They have the ability to boost the 3D construction of tumor growth to amplify tumor cell progression and to help reveal novel anti-cancer treatments. Tumor spheroids can be derived from cancer cell lines and may have the potential to function as mediators for 2D culturing models and for in vivo tissue xenografts. Tumor spheroids produced from permanent cancer cell lines are known to be avascular. They can induce micro-metastasis formation and have the potential to display cancer-like microenvironment features (e.g., cell-cell, cell-ECM exchanges, local nutrients transportation, gases, and growth factors). The term “spheroid” as used herein also encompasses tumour spheroids, also referred to as tumoroids herein. In general, organoids can be referred to as cells grown in 3D to form structural units that partially resemble the organ, both in structure and function.

Organoids are 3D-derived stem cell models, either from embryonic or adult stem cells. Organoids can exhibit self-regulation potential, phenotypic traits of the organ they are grown from, and physiological modelling of the environment through genomic alteration. Furthermore, they can mimic the in vivo 3D architecture and have the capacity of multi-lineage differentiation of different types of tissues (e.g., designing airway organoids from lung-derived stem cell to study lung physiology, patterning, and lung morphogenesis).

The cell clusters described herein are typically cultured in suspension (they are suspended in the cell culture medium). In other words, they are do not adhere to the cell culture vessel in which they are being cultured. The cell culture systems described herein may therefore be referred to as cell cluster-based cell culture systems, wherein the cell clusters (e.g. cells on microcarriers, the spheroids, the organoids, the tissues etc) are in suspension during the culture period.

The cell clusters described herein typically comprise adherent cells. The term “adherent cells” as used herein refers to a homogenous or heterogeneous population of cells which are anchorage dependent, i.e. which require attachment to an artificial support or to other cells (e.g. in the form of a cell cluster) in order to grow in vitro. Suitably, in the context of the present disclosure, the artificial support may be a microcarrier.

The methods of the invention enable the user to reduce aggregation of the cell clusters (e.g. cell-populated microcarriers, spheroids etc) during cell culture. In this context, by “aggregation” it is meant the formation of one or more groups (aggregates) of cell clusters, where a group (aggregate) may contain two or more cell clusters which would otherwise be present individually in the cell culture. For example “aggregation” can refer to the formation of one or more groups (aggregates) of cell-populated microcarriers, where a group (aggregate) may contain two or more cell-populated microcarriers which would otherwise be present individually in the cell culture. In another example, “aggregation” can refer to the formation of one or more groups (aggregates) of spheroids, where a group (aggregate) may contain two or more spheroids which would otherwise be present individually in the cell culture. In another example, “aggregation” can refer to the formation of one or more groups (aggregates) of organoids (or tissues), where a group (aggregate) may contain two or more organoids (or tissues) which would otherwise be present individually in the cell culture. Aggregation in this context typically occurs due to inter-cell cluster interactions. Aggregation is also referred to as “clumping” or “inter-cell cluster aggregation” herein.

In the context of the present disclosure, the term “cell-mediated aggregation” refers to the aggregation of cell clusters as a result of cell—cell interactions (e.g. between the cells of two or more distinct cell clusters to induce clumping), cell-ECM interactions (e.g. between the ECM of one cell cluster and the cells of a distinct cell cluster) and/or cell-microcarrier interactions.

Suitably, the cell—cell interactions may be between cells cultured on (or adhered to) different artificial supports. Suitably, the cell—cell interactions may be between cells cultured on (or adhered to) different microcarriers, thereby causing aggregation of the microcarriers. Cell-cell interaction may occur when cells on different microcarriers adhere to or touch each other (i.e. are interconnected) in a manner that forms an aggregate of the cell-populated microcarriers. As used herein, “interconnected” refers to cells that are in direct contact with each other and are physically connected e.g. by intercellular connections (e.g. by one or more cell junction(s) (also known as intercellular bridge(s)). Cell junctions are made up of multiprotein complexes that provide contact between neighboring cells or between a cell and the extracellular matrix. Cell junctions are especially abundant in epithelial tissues. Cell junctions enable communication between neighbouring cells.

In another example, the cell—cell interactions may be between cells from different cell clusters that do not comprise an artificial support, thereby causing aggregation of the cell clusters. For example, cell-cell interaction may occur when cells on different spheroids adhere to or touch each other (i.e. are interconnected) in a manner that forms an aggregate of the spheroids. In another example, cell-cell interaction may occur when cells on different organoids (or tissues) adhere to or touch each other (i.e. are interconnected) in a manner that forms an aggregate of the organoids (or tissues).

Suitably, cell-microcarrier interactions may occur between a cell adhered to (or cultured on) a first microcarrier and interacting with a second microcarrier, thus facilitating or maintaining aggregate formation.

In some examples, the cells in a cell cluster are all of the same type. For example, they may all be brain cells, muscle cells or heart cells. In other examples, the cells in a cell cluster are all from the same lineage, e.g. all haematopoietic precursor cells. In some examples, the cells are stem cells, for example, neural stem cells or embryonic stem cells.

The cell cluster-based cell culture systems as described herein may be used in continuous bioprocessing systems.

Accordingly, in one example, a cell cluster comprises homogeneous or heterogeneous cell types. By “cell growth” it is meant the division, or proliferation, of the seeded cells.

An “artificial support” as referred to herein is any surface, which is suitable for supporting biological material, for example a cell, cell population, cell culture, tissue, or a fluid or other biological material or composition, for example as described herein. A support may be referred to as a surface, may be suitable for hosting a process or reaction, for example, growth and development of a cell or organism, cell population, cell culture, or tissue. In the context of the present disclosure, the artificial support may be a microcarrier. A “microcarrier” is a particle, suitable for supporting the adherence and/or growth of cells thereon. Microcarriers with cells adhered to (or cultured on) them are referred to herein as cell-populated microcarriers.

A “gel” is a semi-solid, jelly like substance, which does not flow when in the solid state. A gel comprises a 3D cross linked network which provides the gel its semi-solid structure. A gel may be a hydrogel, which comprises a network of insoluble but hydrophilic polymer chains. A gel may be defined in terms of its viscoelasticity or rheological properties.

A “buffer” is a solution which can resist significant changes in pH upon the addition of small amounts of acid or alkali. A buffer is a mixture of a weak acid and its conjugate base, or a weak base and its conjugate acid.

As used herein, “cell viability” refers to the ability of a cell to remain metabolically active for growth and function.

The term “culturing” as used herein refers to maintaining the cells in cell culture for a period of time under conditions suitable for growth. This period may be referred to herein as a “culture period”.

As used herein, “confluency” in the context of a cell cluster comprising an artificial support, refers to the percentage of the support (e.g. microcarrier) which is covered by cells (e.g. adherent cells). 50% confluency means that half of the support (e.g. microcarrier) is covered with cells (e.g. adherent cells). 100% confluency, or fully confluent, means that all of the support (e.g. microcarrier) is covered with cells (e.g. adherent cells). Over-confluency means that there is no available space on the support (e.g. microcarrier) for new cells.

In the context of a cell cluster that does not comprise an artificial support, such as a spheroid, an organoid or a tissue, cell density or cell cluster size is more relevant. Means for determining cell density and size are well known in the art and are described elsewhere herein.

The term “cell culture medium” as used herein refers to a nutritive solution for cultivating live cells so as to allow the cell to proliferate.

The term “exogenous protease” as used herein refers to a protease that is added to the cell culture media (i.e. the cell culture media has been supplemented with the protease). It refers to a protease that has not been expressed by the cells that are present in the cell culture medium (these proteases would be considered as endogenous proteases herein). It is therefore a protease from an external source (it is not naturally produced in this quantity by the cells in the cell culture medium). For the avoidance of doubt, the cells in the cell culture media may be capable of producing these proteases as well, however, the proteases referred to as exogenous proteases herein are those that are added to the cell culture media from an external source. An exogenous protease may also be referred to as a cell culture media supplement or a supplemented protease herein. Suitably, in the context of the present disclosure, the exogenous protease is an exogenous metalloprotease and/or a cysteine protease.

The term “metalloprotease” as used herein refers to a protease having one or more metal ions in the binding/active site. Examples of a metalloprotease include collagenase and/or dispase.

The term “cysteine protease” is intended to describe a protease that possesses a highly reactive thiol group of a cysteine residue at the catalytic site of the enzyme. Cysteine proteases are known in the art and may be referred to herein as “thiol proteases” or “sulfhydryl proteases”. Many superfamilies of cysteine proteases are known in the art. Suitably, in the context of the present disclosure, the cysteine proteases may be of the CA clan (also sometimes referred to as Papain-like proteases). Papain-like proteases share a common catalytic dyad active site featuring a cysteine amino acid residue that acts as a nucleophile. Suitably, the cysteine protease of the CA clan may be selected from the group consisting of ficin, papain, bromelain, cathepsin K and calpain. More suitably, the cysteine protease of the CA clan may be ficin, papain, and/or bromelain. More suitably, the cysteine protease of the CA clan may be ficin and/or papain. The term “CA clan” is based on the MEROPS classification scheme (https://www.ebi.ac.uk/merops/).

Suitably papain may be formulated as Performase®. Performase® is powder preparation of purified papain, standardized with Maltodextrin. Papain is an enzyme that is extracted from the green unripe fruit of the papaya tree.

Ficin, also sometimes referred to as ficain, is a proteolytic enzyme typically extracted from the latex sap from the stems, leaves, and unripe fruit of the American wild fig tree Ficus insipida.

The term “contacting” refers to causing two or more items to come into contact with each other. The items may be two or more of cells, a support, and/or a cell culture medium, suitably as defined herein. Contacting may include causing or placing two or more of the above items into close physical relationship and/or touching with each other. Contacting may sometimes be referred to herein as “exposing”, for example exposing cells to a cysteine protease shall be therefore understood as contacting cells with a cysteine protease.

By “seeding” cells it is meant the application of an initial cell population to the cell culture media or support. “Seeded cells” are the cells initially applied to the support.

The term “detach” or “detached” with reference to a cell on an artificial support (e.g. a microcarrier) means separation of the cell from the support (e.g. microcarrier), so that it is no longer anchored or adhered to the support (e.g. microcarrier).

The term “detach” or “detached” with reference to a cell cluster (e.g. a spheroid, organoid or tissue) that does not comprise an artificial support means separation of the cell from the cell cluster (e.g. a spheroid, organoid or tissue), so that it is no longer anchored or adhered to the cell cluster (e.g. a spheroid, organoid or tissue).

“Frequency of application” as used herein refers to the number of times a reagent, in particular the protease (such as a collagenase and/or dispase), is added to the cell culture medium, either prior to and/or during a cell culture period.

“Log phase of growth” is the logarithmic or exponential phase of cell growth where the cells are actively proliferating and the cell density is increasing. The log phase typically follows the lag phase and is prior to the stationary phase where growth slows or stops.

The term “suspension” or “suspension culture” is a type of cell culture in which cells are grown in small cell clusters or on artificial supports such as microcarriers, in an agitated growth medium.

The term “cell cluster based cell culture system” means a cell culture system which cultures cells in cell clusters in suspension, as opposed to, for example, on a surface of a cell culture flask. The term “microcarrier based cell culture system” means a cell culture system which utilises microcarriers to culture cells thereon, as opposed to, for example, a surface of a cell culture flask. The term “spheroid based cell culture system” means a cell culture system which cultures cells in spheroids, as opposed to, for example, a surface of a cell culture flask. The term “organoid (or tissue) based cell culture system” means a cell culture system which cultures cells in organoids (or tissues), as opposed to, for example, a surface of a cell culture flask.

The term “reducing” as used herein in the context of reducing aggregation in a cell cluster-based cell culture system, refers to decreasing the proportion or number or size of aggregates formed within the cell culture as compared to a suitable control. The decrease may be for example by at least 20%, at least 30%, at least 40%, at least 50%, at least 60%, at least 70%, at least 80%, at least 90%, or more as compared to a control. Suitably, the decrease may be by about 100% as compared to a control. In the context of the present disclosure, the control may be the level of aggregation in a method comprising the step of: (i) culturing a cell population comprising two or more cell clusters in a cell culture medium for a culture period, wherein the cell culture medium does not comprise an exogenous protease.

The methods can also be referred to herein as “reducing” clumping in a cell cluster-based cell culture system.

### DETAILED DESCRIPTION

The present invention provides a method of reducing aggregation in a cell cluster-based cell culture system, the method comprising the step of (i) culturing a cell population comprising two or more cell clusters in a cell culture medium for a culture period, wherein the cell culture medium comprises an exogenous protease, wherein the exogenous protease is an exogenous metalloprotease and/or cysteine protease.

Suitably, aggregation of two or more cell clusters in the cell culture may be cell-mediated aggregation.

The present invention is based upon the surprising finding that aggregation can be reduced in a cell cluster based cell culture system by reducing overgrowth of cells and/or by preventing bridging between cell clusters in the cell culture medium. The invention is based upon providing in the cell culture medium, suitably during the growth phase, a metalloprotease (such as a collagenase and/or dispase) and/or a cysteine protease (such as papain and/or ficin) which disrupts cell-cell interactions between different cell clusters within the cell culture and/or detaches a proportion of cells from the cell cluster to prevent overgrowth. The present invention therefore provides a method for reducing clumping during cell culture of e.g. cell-populated microcarriers, spheroids, organoids or tissues in suspension culture. The method of the invention also provides a means for controlling the confluency of cells on an artificial support such as a microcarrier. As a result, the cells can be prevented from reaching high confluency and/or overgrowth. The invention therefore provides a new means for reducing or indeed preventing aggregation (clumping) during cell culture. Surprisingly, the inventors found that other classes of proteases, such as serine proteases (for example trypsin), do not have the same utility in the context of the present invention due to resulting in bulk detachment. Without being wishing to be bound by this hypothesis, the inventors believe that is due to metalloproteases and cysteine proteases having a different mechanism of action as compared to serine proteases. Specifically, serine proteases may transmit intracellular signals, whereas metalloproteases and cysteine proteases act on the extracellular matrix, not with/on the cells and do not directly trigger any intracellular response.

The presence of a metalloprotease and/or cysteine protease in the cell culture medium reduces aggregation of cell clusters (such as aggregation of cell-populated microcarriers, spheroids, organoids, tissues, as appropriate). In the presence of an exogenous metalloprotease and/or cysteine protease in the cell culture medium, the proportion of cell clusters which aggregate together may suitably be 25% or less, 20% or less, 15% or less, 10% or less, or 5% or less of the total number of cell clusters in the cell culture medium. Indeed the presence of aggregates in a cell culture medium may be substantially 0%, meaning that the protease essentially prevents aggregation of cell clusters. Thus, a desired proportion of the cell clusters will remain in suspension in the cell culture medium and will not become attached to other cell clusters present in the medium. Suitably, the presence of a metalloprotease and/or a cysteine protease in the culture medium also serves to reduce or prevent binding or aggregation of the cell clusters on a surface of a cell culture system, such as a bioreactor. Suitably, the present invention provides for a cell culture in a bioreactor where the cell clusters are maintained as a homogenous (suspended) population in the cell culture medium.

The method of the invention has the advantage of increasing the growth capacity of the cell culture, by reducing or preventing aggregation which may limit access of cells to nutrients and/or gases and result in a decrease or limitation of growth. The method of the invention also improves harvesting of cells from the microcarriers, by avoiding overgrowth, cell clumping, reducing shear, and/or loss of viable cells due to aggregation.

A method of the invention may comprise one or more steps prior to step i). A method may comprise for example, obtaining a cell population from a suitable source.

The present invention is particularly suitable for the growth of any adherent cell. Anchorage dependent cells are typically derived from a multi-cellular organism. Suitably, the adherent cells may be mammalian cells, for example human, mice, rat, rabbit, dog, cat, cow, pig, chicken, goat, horse, etc. Mammalian cells may be derived from any suitable tissue, for example adrenal, bladder, blood vessel, bone, bone marrow, brain, cartilage, cervical, corneal, endometrial, oesophageal, gastrointestinal, immune system (e.g., T lymphocytes, B lymphocytes, leukocytes, macrophages, and dendritic cells), liver, lung, lymphatic, muscle (e.g., cardiac muscle), neural, ovarian, pancreatic (e.g., islet cells), pituitary, prostate, renal, salivary, skin, tendon, testicular, and thyroid. In some embodiments, the cells are mammalian cells (e.g., human). The adherent cells may be non-mammalian cells, such as insect cells, bird cells, or fish cells. The cell may be a prokaryotic cell, for example a fungal cell or a bacterial cell. The adherent cells may be plant cells. Suitably, the adherent cells may be primary cells or immortalised cells. Merely by way of example, primary cells may be selected from the group consisting of myocytes, cardiomyocytes, epithelial cells, fibroblasts, keratinocytes, melanocytes, endothelial cells, osteoblasts, chondrocytes, adipocytes, and mesenchymal stem cells. Merely by way of example, immortalised cells may be selected from the group consisting of Hela cells, HEK 293 cells, 3T3 cells, A549 cells, Vero cells, CHO cells, OK cells, C2C12 and PTK2 cells. The cell may be a disease cell or a disease model cell, for example a cancer cell or a cell in a hyperproliferative state. In one example, the cell population comprises cells selected from the group consisting of: skin, muscle, cervical, breast, and prostate cells.

Suitable cells are described elsewhere herein, and the skilled person would understand how to obtain such a cell population. For example, the method may comprise obtaining a suitable tissue, and isolating the cells therefrom.

The cells may be cultured as an adherent cell population on an artificial support such as a microcarrier.

Suitably, a cell population on a microcarrier may be at least, or no more than, 50%, 60%, 70%, 80%, 90% or 100% confluent, meaning that at least, or no more than, 50%, 60%, 70%, 80%, 90% or 100% of the surface area of the microcarrier is occupied by cells. Where substantially all of the surface area of a microcarrier is occupied by cells, the cell layer may be referred to as being confluent. In one example, the presence of a metalloprotease and/or a cysteine protease in the cell culture medium prevents a cell layer on a microcarrier from becoming confluent or over-confluent or maintains the confluency at the desired level.

Suitably, the cell layer is maintained at 80%-100% confluency during the cell culture period. Suitably, the confluency on a microcarrier does not exceed 50%, 60%, 70%, 80%, 90%, 95% or 100%. The degree of confluency on any two or more microcarriers in a cell culture system of the present invention may be the same or may be different. The degree of confluency on any two or more microcarriers in a cell culture system of the present invention may fall within a range of, for example, 5% to 95% confluency, for example 80% to 100% confluency.

In a defined cell culture period, cells may be detached continuously, meaning that under the method of the invention, the stationary phase of growth is not reached as the metalloprotease (for example collagenase and/or dispase) and/or cysteine protease (such as papain and/or ficin) present in the cell media continually detaches the cells from the cell clusters in the cell culture medium.

The method of the invention is associated with a number of advantages. For example, the method enables an increase in the yield of cells from the cell culture, by maintaining the cells in the log phase of growth for a longer period of time. In addition, the method provides advantages of reducing the amount of resources required to produce a desired yield, and consequently the resource or carbon footprint.

When a microcarrier is used in the context of the invention, the layer of cells in contact with the microcarrier may be a monolayer of cells (meaning that the layer is one cell deep), or more than one layer of cells (two or more cells deep). The cells may comprise one type of cell, or two or more different cell types.

Prior to step i), a method of the present invention may comprise one or more of culturing, maintaining, passaging, separating, and/or isolating the cell population. Where a cell is cultured or maintained in a cell culture medium prior to step i), this may be in the absence of an exogenous protease.

The invention comprises culturing a cell population comprising a plurality of cell clusters. Each cell cluster may be a cell-populated microcarrier. Reference to a microcarrier may include a single microcarrier or a population of microcarriers. A population may comprise two or more microcarriers. The microcarriers in a single population (in a single cell culture) may all be identical, or may be different, for example in terms of size, shape, material, porosity, density, and/or surface modification. A suitable microcarrier or combination of microcarriers for use in the present invention may be selected by the skilled person based upon factors including the type of cells to be cultured, and the desired outcome of the culture. Where necessary, a microcarrier may be modified or adapted to support or improve adherence of cells thereto.

The microcarrier may be provided in a suitable growth chamber or vessel.

Before use, the microcarrier and/or suitable growth chamber or vessel may be typically sterilized. Sterilization may be performed, for example, by gamma-irradiation, by autoclave, by washing with alcohol or by ethylene oxide (ETO) gas treatment.

A microcarrier may be a particle, for example a bead. A microcarrier may be spherical or ovoid. A microcarrier may comprise a planar surface, upon which a cell population may be cultured.

A microcarrier may be rigid or may be elastic. A microcarrier may be porous or may be non-permeable. A material which is not naturally porous can be made porous by methods available to the skilled person, for example sintering, etching, leaching, lithography, or laser micromachining. An example of a porous support may be a gel or a mesh. A microcarrier may be edible.

In one example, the microcarriers comprises or consists of DEAE-dextran, glass, polystyrene, plastic, PEG, acrylamide, or a natural polymer such as gelatin, collagen, chitin and its derivatives, and cellulose, hyaluronic acid, alginate, PLGA, or gel, or any combination thereof.

It will be appreciated that all or part of a microcarrier may be made from or treated with a material that facilitates cell adhesion. A microcarrier may be made of a material which supports cell adhesion, or all or part of the surface thereof may be modified to support cell adhesion. A material that facilitates cell adhesion may be selected from the group consisting of a polyester, a polypropylene, a polyalkylene, a polyfluoro chloroethylene, a polyvinyl chloride, a polyvinyl fluoride resin, a polystyrene, a polysulfone, a polyurethane, a polyethylene terephthalate, a cellulose, a glass fiber, a ceramic particle, a matrigel, an extracellular matrix component, a collagen, a poly-L-lactic acid, a dextran, an inert metal fiber, silica, natron glass, borosilicate glass, chitosan, or a vegetable sponge. Suitably, the cellulose may be cellulose acetate. The extracellular matrix component may be one or more of fibronectin, vitronectin, chondronectin, or laminin. Suitably, the adherent material is electrostatically charged. Suitably, the adherent material may be collagen or gelatin. Suitably, the adherent material may comprise a peptide amphiphile (PA), such as that described in Miotto et al, Developing a Continuous Bioprocessing Approach to Stromal Manufacture, ACS Applied Materials & Interfaces 2017 9 (47), 41131-41142. Adherent cells may attach to the surface of a microcarrier through an anchorage substrate such as integrin or other cell receptor. A microcarrier may comprise a non-adherent portion.

Microcarriers of the same material can differ in their porosity, specific gravity, optical properties, presence of animal components, and surface chemistries or modifications. Surface chemistries can include the presence of extracellular matrix proteins, functional groups, recombinant proteins, peptides, and positively or negatively charged molecules, added through conjugation, co-polymerization, plasma treatment or grafting to the surface of a microcarrier.

A microcarrier may have a diameter in the range from 50 μm to 1 mm, suitably around 100 μm in diameter. It will be appreciated that the size and composition of the microcarrier may be dictated by the type of cells to be cultured on the microcarrier. Selecting appropriate microcarriers is well within the ability of a person skilled in the art.

The invention comprises culturing a cell population comprising a plurality of cell clusters. Each cell cluster may be a spheroid. Reference to a spheroid may include a single spheroid or a population of spheroids. A population may comprise two or more spheroids. The spheroids in a single population (in a single cell culture) may all be identical, or may be different, for example in terms of size, shape and/or density. The spheroid may be provided in a suitable growth chamber or vessel.

In an alternative example, each cell cluster may be an organoid (or a tissue). Reference to an organoid (or a tissue) may include a single organoid (or a tissue) or a population of organoids (or a tissue). A population may comprise two or more organoids (or tissues). The organoids (or tissues) in a single population (in a single cell culture) may all be identical, or may be different, for example in terms of size, shape and/or density. The organoid (or tissue) may be provided in a suitable growth chamber or vessel.

The method of the present invention may be carried out in a growth chamber. As would be clear to a person of skill in the art, a “growth chamber” refers to any suitable chamber or vessel that is suitable for the growth of cells. It will be appreciated that the growth chamber may comprise microcarriers and cell culture medium. Suitably, the growth chamber may be a bioreactor.

A bioreactor is any suitable chamber that is suitable for containing a cell cluster as described herein, suitably in suspension. A bioreactor may therefore have any suitable type of surface and any suitable type of geometry. Suitable bioreactors may therefore include, but are not limited to hollow fibre, stirred tank, air-lift, bubble column or fluidised bed bioreactor, packed bed bioreactor or a flexible bag. A bioreactor may be a liquid phase, gas phase, or hybrid bioreactor. A bioreactor may be single use or multiple use. It may be a benchtop bioreactor or may be an industrial bioreactor. A bioreactor may be horizontal or vertical and may have a stirrer or be subject to wave or rocking motion. Accordingly, it is clear that any suitable growth chamber (e.g. a bioreactor) may also be used.

Prior to step i) a suitable cell culture medium may be selected. The cell culture medium may be a complete formulation, i.e., a cell culture medium that requires no supplementation to culture cells, or may be an incomplete formulation, i.e., a cell culture medium that requires supplementation or may be a medium that may supplement an incomplete formulation or in the case of a complete formulation, may improve culture or culture results. Various cell culture media will be known to those skilled in the art, who will also appreciate that the type of cells to be cultured may dictate the type of culture medium to be used. Suitably, a cell culture media is selected which does not substantially affect the activity of the selected metalloprotease (such as collagenase and/or dispase), and/or cysteine protease (such as papain and/or ficin).

Merely by way of example and not limitation, the cell culture medium may be selected from the group consisting of Dulbecco's Modified Eagle's Medium (DMEM), Ham's F-12 (F-12), Leibovitz's L-15 medium, RPMI-1640, Mesencult™ Basal Medium, Minimal Essential Medium (MEM), Basal Medium Eagle (BME), Ham's F-10, aMinimal Essential Medium (αMEM), Glasgow's Minimal Essential Medium (G-MEM), and Iscove's Modified Dulbecco's Medium (IMDM), or any combination thereof. Other media that are commercially available (e.g., from Thermo Fisher Scientific, Waltham, MA) or that are otherwise known in the art can be equivalently used in the context of this disclosure. Again, only by way of example, the media may be selected from the group consisting of 293 SFM, CD-CHO medium, VP SFM, BGJb medium, Brinster's BMOC-3 medium, cell culture freezing medium, CMRL media, EHAA medium, eRDF medium, Fischer's medium, Gamborg's B-5 medium, GLUTAMAX™ supplemented media, Grace's insect cell media, HEPES buffered media, Richter's MEM, IPL-41 insect cell medium, McCoy's 5A media, MCDB 131 medium, Media 199, Modified Eagle's Medium (MEM), Medium NCTC-109, Schneider's Drosophila medium, TC-100 insect medium, Waymouth's MB 752/1 media, William's Media E, protein free hybridoma medium II (PFHM II), AIM V media, Keratinocyte SFM, defined Keratinocyte SFM, STEMPRO® SFM, STEMPRO® complete methylcellulose medium, HepatoZYME-SFM, Neurobasal™ medium, Neurobasal-A medium, Hibernate™ A medium, Hibernate E medium, Endothelial SFM, Human Endothelial SFM, Hybridoma SFM, PFHM II, Sf 900 medium, Sf 900 II SFM, EXPRESS FIVE® medium, CHO-S-SFM, AMINOMAX-II complete medium, AMINOMAX-C100 complete medium, AMINOMAX-C140 basal medium, PUB-MAX™ karyotyping medium, KARYOMAX bone marrow karyotyping medium, and KNOCKOUT D-MEM, or any combination thereof.

Suitably the cell culture medium may be serum-free. Suitably the cell culture medium may be glucose free.

Alternatively, the cell culture medium may comprise serum. Appropriate types and amounts of serum are known e.g. 1% FBS may be used.

Suitably, the cell culture medium may comprise one or more additional reagents, as required. These may be selected from, but are not limited to, the group consisting of an antibiotic, buffer, growth factor, hormone, nutritional supplement, indicator, and essential metals and minerals.

In a next step prior to step i), the cells may be seeded on a microcarrier. Suitably, the cells are seeded onto the microcarrier after the microcarrier is incubated with the cell culture media. Methods of cell seeding are known and available to the person skilled in the art and may be adapted according to the cell type and support. Two or more microcarriers may be seeded simultaneously. The initial cell seeding density has to be efficient while allowing optimal cell proliferation within the microcarriers. The number of the cells to be seeded further depends on porosity of the scaffold material and any liquid absorption capability. For a porous microcarrier, the greater the porosity, the larger the number of cells that can be seeded. In some embodiments, the number of cells per g support (dry weight) is in the range of 2×10⁶ to 50×10⁶ cells. In addition, the porosity of the scaffold and the internal organization of support fibers contribute to the retention of the cells within and on the support. The method may comprise more than one sequential seeding steps on a support.

The metalloprotease (such as collagenase and/or dispase) and/or cysteine protease (such as papain and/or ficin) may be provided in the cell culture medium, prior to the application of the cell culture medium to the cell culture. Therefore, the cell culture medium comprising a metalloprotease (such as collagenase and/or dispase) and/or cysteine protease (such as papain and/or ficin) may be added to the support or to the growth chamber itself. Alternatively, the metalloprotease (such as collagenase and/or dispase) and/or cysteine protease (such as papain and/or ficin) may be provided separately to the cell culture medium. Suitably, the metalloprotease (such as collagenase and/or dispase) and/or cysteine protease (such as papain and/or ficin) is added to the cell culture prior to initiation of the culture period. Therefore metalloprotease (such as collagenase and/or dispase) and/or cysteine protease (such as papain and/or ficin) may be added to the cell culture during the lag phase of cell growth.

Alternatively, or additionally, the metalloprotease (such as collagenase and/or dispase) and/or cysteine protease (such as papain and/or ficin) may be added to the cell culture towards the end of log phase of cell growth, or when the cells reach a confluency of at least 50%, at least 60%, at least 70% etc. In some examples, the metalloprotease (such as collagenase and/or dispase) and/or cysteine protease (such as papain and/or ficin) may be added to the cell culture when the cells reach a confluency of at least 80% or 85%.

Additional metalloprotease (such as collagenase and/or dispase) and/or cysteine protease (such as papain and/or ficin) may be added to the cell culture during the culture period. Supplemental metalloprotease (such as collagenase and/or dispase) and/or cysteine protease (such as papain and/or ficin) may be added to the cell culture once or more than once during a culture period. For example, supplemental metalloprotease (such as collagenase and/or dispase) and/or cysteine protease (such as papain and/or ficin) may be added to the cell culture 2, 3, 4, 5, 6, 7, 8, 9, 10, 11, 12, 13, 14, 15 times or more during a cell culture period. Supplemental metalloprotease (such as collagenase and/or dispase) and/or cysteine protease (such as papain and/or ficin) may be added to the cell culture at regular or irregular intervals. Regular intervals may, for example, be every hour, 2 hours, 3 hours, 4 hours, 5 hours 6, hours, 7 hours, 8 hours, 9 hours, 10 hours, 11 hours, 12 hours, 24 hours, 36 hours, 48 hours, or 72 hours. Regular administration of additional metalloprotease (such as collagenase and/or dispase) and/or cysteine protease (such as papain and/or ficin) may be referred to as “pulsing”. If supplemental metalloprotease (such as collagenase and/or dispase) and/or cysteine protease (such as papain and/or ficin) is added in at irregular intervals, these may depend upon factors such as the growth rate of the cells, the rate of detachment, the degree of confluency on the microcarriers, and/or the concentration of metalloprotease (such as collagenase and/or dispase) and/or cysteine protease (such as papain and/or ficin) in the cell culture media. Supplemental metalloprotease (such as collagenase and/or dispase) and/or cysteine protease (such as papain and/or ficin) may be provided to the cell culture where the rate of detachment is below the rate growth, for example by 10%, 20%, 30%, 40% or 50% or more.

Supplemental metalloprotease (such as collagenase and/or dispase) and/or cysteine protease (such as papain and/or ficin) may be added with cell culture media. In such an embodiment, an amount of the existing cell culture media may be removed, to maintain the desired concentration of the protease (such as collagenase and/or dispase). Suitably, removal or addition of cell culture media does not amount to washing of the cells and does not alter the growth phase of the cells when in the log phase. In a cell culture period, the cell culture media is not wholly removed, and wash media is not added to the cell culture.

The cell culture is maintained under suitable cell culture conditions, at least for a cell culture period. Suitable cell culture conditions for different cell types will be known to a person skilled in the art. Typical growth conditions are 37° C., at a relative humidity of 95% and CO₂ availability of 5%. The pH may be maintained at 7-7.4.

The cell culture may be maintained under static or dynamic conditions. In a dynamic system, movement is applied to the cell culture (for example by placing on a rocker) to allow movement of the cell culture media. Agitation has the effect of maintaining cells and/or cell clusters in suspension in the cell culture medium. In a static system, there is no mechanism provided for movement of the cell culture media. In such as system, the cell clusters may form a static bed.

The cell culture period may be defined as comprising all or part of the log phase of growth of the cell population or may be defined as being at least 24 hours. The culture period may be at least 24 hours and may comprise all or part of the log phase of growth of the cell population. A log phase may be any period between the lag phase and the stationary phase, where the cells in the culture are actively proliferating. Suitably, the culture period includes all or part of the log phase, suitably at least 40%, 50%, 60%, 70%, 80% or at least 90% of the log phase. Suitably, the culture period does not include all or part of the lag phase or the stationary phase. The growth phase of the cell culture may be determined using any suitable method, for example visual or analytical methods (such as cell counting, DAPI staining etc).

Suitably, a cell culture may be at least 24 hours (about 1 day), at least about 36 hours, at least about 48 hours (about 2 days), at least about 60 hours, at least about 72 hours (about 3 days), or more. Suitably, cell culture may be for at least about 84 hours, at least about 96 (about 4 days) hours, at least about 108 hours, at least about 120 hours (about 5 days), at least about 132 hours, at least about 144 hours (about 6 days), at least about 156 hours, at least about 168 hours (about 7 days), at least about 180 hours, at least about 192 hours (about 8 days), at least about 204 hours, at least about 216 hours (about 9 days), or more. Suitably, cell culture may be from about 1 day to about 9 days, for example from about 2 days to about 8 days, or from about 3 days to about 7 days.

Suitably, a cell culture may be at least about 7 days, at least about 14 days, at least about 21 days, at least about 28 days, at least about 35 days, or more. Suitably, cell culture may be for at least about 40 days.

Suitably, a cell culture may be at least about 4 weeks, at least about 8 weeks, at least about 12 weeks, at least about 16 weeks, at least about 20 weeks, or more. Suitably, a cell culture may be at least about 6 months, at least about 8 months, at least about 10 months, at least about 12 months, or more.

In a method of the invention, suitably the cells (e.g. on the microcarrier) do not reach confluency during a cell culture period, or suitably do not become over-confluent. Suitably, a method of the present invention enables the cells to maintain a confluency of about 50-100%, more suitably 60-95%, more suitably 80-95%, or any range or integer therebetween, for example about 50, 55, 60, 65, 70, 75, 80, 85, 90 or 95% during a cell culture period. Suitably, the confluency is maintained below 100% during a cell culture period. In another example the confluency may be maintained at or below about 100% during the cell culture period.

The confluency may be maintained at a level as described herein by the use of the metalloprotease (such as collagenase and/or dispase) and/or cysteine protease (such as papain and/or ficin) in the cell culture medium during the cell culture period, which allows for continuous detachment of the cells (e.g. from the support or from other adherent cells e.g. from a cell aggregate) as the cells as proliferating and the population is growing.

In the methods of the invention, the metalloprotease (such as collagenase and/or dispase) and/or cysteine protease (such as papain and/or ficin) may continuously detach a proportion of the cells from the cell clusters. In this context, “continuously detaches a proportion of the cells” refers to continually removing a proportion of the cells in the cell population (e.g. removing a proportion of the cell population on a microcarrier or removing a proportion of the cell population in a spheroid, organoid or tissue). The detachment therefore occurs gradually over time and is not one discrete (bulk detachment) event.

As would be clear to a person of skill in the art, continuous detachment occurs over a period of time (over the culture period, which is typically more than 24 hours). Accordingly, the metalloprotease (such as collagenase and/or dispase) and/or cysteine protease (such as papain and/or ficin) continuously detaches a proportion of the cells from the cell clusters in the cell population over the culture period. Typically, the methods of the invention comprise detaching adherent cells from the clusters in the cell population (using an exogenous metalloprotease (such as collagenase and/or dispase) and/or cysteine protease (such as papain and/or ficin)) whilst the cell population is undergoing a growth phase (typically whilst the cell population is in log phase). The continuous detachment therefore may serve to maintain the cell population at a confluency or density that enables the cell population to remain in log growth phase over the culture period (or at least avoid the stationary and lag phases of cell growth). This may be particularly useful in the context of continuous bioprocessing systems such as bioreactors. During step (i), the methods of the invention therefore typically maintain the adherent cell population at a confluency of between 40% and 95% (e.g. when adherent on a support). Optimal confluence is discussed elsewhere herein and would be readily determined by a person of skill in the art.

Accordingly, the metalloprotease (such as collagenase and/or dispase) and/or cysteine protease (such as papain and/or ficin) may continuously detach a proportion (e.g. at least 5%, but no more than 60%) of the cells from the cell clusters in the cell population over the culture period.

Accordingly, the metalloprotease (such as collagenase and/or dispase) and/or cysteine protease (such as papain and/or ficin) may continuously detach a proportion (e.g. at least 0.5%, but no more than 7% per hour) of the cells from the cell clusters in the cell population over the culture period.

In the methods of the invention, continuous detachment may maintain the cell confluency or density of the cells in the cell clusters over the culture period (in other words, the cell confluency or density may in some examples increase or decrease by no more than 40% over the culture period).

In the methods of the invention, continuous detachment may maintain the cell confluency or density of the cells in the cell clusters over the culture period (in other words, the cell confluency or density may in some examples increase or decrease by no more than 40% on weekly average for the culture period).

As would be clear to a person of skill in the art, “continuously detaching” does not encompass bulk detachment (which is a term that is well known in the art). In the context of an exogenous metalloprotease (such as collagenase and/or dispase) and/or cysteine protease (such as papain and/or ficin), bulk detachment may be referred to as the detachment of at least 60% of cells in the cell clusters in 30 minutes at 37° C. Step (i) of the methods of the invention therefore do not comprise bulk detachment of the cells in the cell clusters.

Suitably, in a culture period, the rate of detachment exceeds the rate of proliferation.

Suitably, the metalloprotease (such as collagenase and/or dispase) and/or cysteine protease (such as papain and/or ficin) is provided in the cell culture medium at a concentration which allows for the ratio of the rate of detachment of cells (from a cell cluster e.g. from the microcarrier or from other adherent cells e.g. from a spheroid, organoid, tissue) to the rate of growth of the cells to be in the range of 0.3:1 to 1:3. In other words, the rate of detachment may be a third or more of the rate of proliferation. The rate of proliferation may be up to 3 times the rate of detachment. The rate of detachment and rate of proliferation may be determined by, e.g. imaging, bioreactor sensors/probes, analytical methods, all of which are well known in the art.

Suitably, the detached cells, or a proportion of the detached cells, may be harvested at least once during the cell culture period. Suitably, the detached cells, or a proportion of the detached cells, may be harvested more than once during the cell culture period. Harvesting of detached cells may take place every hour, 2 hours, 3 hours, 4 hours, 5 hours 6, hours, 7 hours, 8 hours, 9 hours, 10 hours, 11 hours, 12 hours, 24 hours, 36 hours, 48 hours, or 72 hours. Suitable methods of removing detached cells from the culture include using dead-end or crossflow/tangential fluid flow filtration, a centrifuge, acoustic separation, or a microfluidic based system. Alternatively, detached cells are not harvested, and may remain in the cell culture until after the cell culture period.

Before or after step i) of a method of the invention, the cells may be passaged one or more times. Thus, a method may comprise removing cells from a cell culture and placing said cells onto e.g. a new microcarrier. Suitably, the cells are a population of cells which are detached by a method of the invention.

A method of the invention may additionally comprise monitoring or determining the viability of the cells in the cell culture. Cell viability may be measured through measurement of cell proliferation or metabolic activity. Other methods include flow cytometry and immunohistochemistry. A measure of cell viability may be made after the culture period of step i).

Suitably, in a method of the invention, the metalloprotease (such as collagenase and/or dispase) and/or cysteine protease (such as papain and/or ficin) does not have a substantial detrimental effect of the viability of the cell population (e.g. over a culture period of at least 24 hours, e.g. at least 48 hours, e.g. at least 72 hours). In this context, a substantial detrimental effect may be considered as a 10% decrease in cell viability over the culture period compared to the cell viability observed in the absence of the metalloprotease (such as collagenase and/or dispase) and/or cysteine protease (such as papain and/or ficin).

A method of the invention may additionally comprise monitoring or determining the confluency of cells (e.g. on the support). Cell confluency may be measured using chemical dyes (e.g. thymidine, Alamar blue, XTT or others available in the art), qualitative visual measurement status, or using an image processing method including for example an Olympus CKX53 culture microscope, CKX-CCSW confluency checker software and the Air Fraction output. As described in the examples below, cells may also be imaged and analysed using Cytation 1, loLight, Jiusion USB digital Microscope and ImageJ. Where the method indicates that the cells are more confluent than desired, additional protease (for example collagenase and/or dispase) may be added to the cell culture media to increase the rate of detachment of cells from the cell cluster. Account may be taken of the overall concentration of protease (for example collagenase and/or dispase), to remain within optimal limits for the cell type.

Cell confluency may also be referred to as the density of adhered cells (e.g. on the support or the density of cells in the cell cluster). Cell density may be determined using e.g. microscopy, acoustic resonance densitometry, laser induced fluorescence, fluorescence microscopy, capacitance impedance, turbidity, a biomass permittivity probe, a Raman probe and a cell counter e.g. CCD imaging using Trypan blue. Cell density may also be used to determine the rate of detachment of cells from a cell cluster, as described elsewhere herein.

A method of the invention may include monitoring or determining the growth phase of the cells in culture. This may be performed using visual means, such as checking cell shape.

Any suitable cell imaging method may be used, for example imaging or otherwise analyzing cells, for example fluorimeters, luminometers, cameras, microscopes, plate readers, cell analysers, and confocal imaging systems.

A method of the invention may comprise repeating step i) two or more times.

Suitably, the culture period in step i) does not comprise a step of washing the cells. A washing step may be included after step i). Washing may be performed using any suitable method dependent on the choice of cell (and e.g. the choice of cell cluster). Washing may include aspirating cell culture media and be performed by placing the cells in a physiological buffer.

A method of the invention may include counting the cells.

Suitably, in the cell culture period, substantially all the cells of the cell population do not differentiate. Therefore, substantially all of the cells of the cell population will not show signs of differentiation, for example differentiation markers or changes in cell shape or morphology.

A method of the present invention may further comprise culturing the cells under conditions suitable to allow differentiation.

A method of the present invention may further comprise cryopreservation of harvested cells.

Any suitable source of an exogenous metalloprotease (such as collagenase and/or dispase) and/or cysteine protease (such as papain and/or ficin) may be used in the present invention. Suitably, the metalloprotease (such as collagenase and/or dispase) and/or cysteine protease (such as papain and/or ficin) is compatible with a cell culture media. Suitably, the metalloprotease (such as collagenase and/or dispase) and/or cysteine protease (such as papain and/or ficin) is capable of detaching cells without substantially affecting cell growth or viability.

A metalloprotease (such as collagenase and/or dispase) and/or cysteine protease (such as papain and/or ficin) may be pure or may contain trace amounts (e.g. 15% or less, or 10% or less) of other proteins or enzymes.

In one example, the collagenase type I, and as such may contain other enzymes such as caseinase, clostripain and trypsin. Suitably, the collagenase type I may have more than 125 U/mg of collagenase; more than 200 U/mg of caseinase; less than 4 U/mg of clostripain and less than 0.5 U/mg of tryptic activity.

Where the metalloprotease is a collagenase, it may be that which is obtained from clostridia histolyticum and is also used in human medicine. However, it can also be isolated from other clostridia bacteria or tissues (e.g. Merck Index, No. 2477).

A suitable collagenase may be Gibco™ Collagenase Type I (product code 11500536 from Fisher Scientific) that is isolated from Clostridium histolyticum which contains average levels of collagenase, caseinase, clostripain, and tryptic activities. This is the collagenase type I used in the examples below.

A number of FDA approved collagenases are known and available in the art. These include, for example, Santyl (Smith and Nephew); cellulite (Qwo, Endo International), and Xiaflex (Endo International).

Suitably, a single metalloprotease (i.e. collagenase or dispase) or cysteine protease (i.e. papain or ficin) may be used in a cell culture, in a single cell culture period. Alternatively, a combination of two or more proteases that are metalloproteases or cysteine protease may be provided in a cell culture, in a single cell culture period. Merely by way of example one metalloprotease (i.e. collagenase or dispase) and one cysteine protease (i.e. papain or ficin) may be used in cell culture in a single cell culture period, or combination of two or more metalloproteases (such as collagenase and dispase) or cysteine protease (i.e. papain and ficin) may be used in cell culture in a single cell culture period. Combinations of metalloproteases and cysteine proteases may also be used in cell culture in a single cell culture period.

As would be clear to a person of skill in the art, any suitable concentration or amount of metalloprotease (such as collagenase and/or dispase) and/or cysteine protease (such as papain and/or ficin) may be used, provided that the protease reduces aggregation of the cell clusters within the cell population during the culture period. A person of skill in the art can readily determine suitable concentrations and amounts based on the disclosure herein and their common general knowledge. The exact amount to be used may depend on a number of factors e.g. the type of cells, the cell culture medium, whether static or dynamic conditions are used, whether cell detachment is desirable or not etc. Some non-limiting examples are provided herein. For example, it is shown that a higher concentration of collagenase was needed for static culture compared to dynamic culture in a flat system, under serum free conditions. Even higher concentrations were needed when microcarriers were used. Further increases in concentration have also been observed when serum was present in the cell culture media (flat-static-steady state continuous detachment achieved with 9 to 20 U/mL of collagenase type I; compared to flat-dynamic-steady state continuous detachment achieved with 5 to 10 U/mL; with bulk detachment requiring 4200 U/mL (20 mg/mL)-data not shown). A suitable concentration may therefore be determined by a person of skill in the art. Suitably, where collagenase is used, it may be provided in a cell culture at or below 500 U/mL. Suitably, it may be provided in a cell culture at or below 215 U/mL. Suitably, the collagenase may be provided in a cell culture at or below 120 U/mL. Suitably, the collagenase may be provided in a cell culture at or below 110 U/mL or at or below 50 U/mL.

Suitably, where the collagenase is collagenase type I, it may be provided in a cell culture at or below 215 U/mL. Suitably, the collagenase type I may be provided in a cell culture at or below 120 U/mL. Suitably, the collagenase type I may be provided in the cell culture at or below 110 U/mL. Suitably, the collagenase type I may be provided in the cell culture at or below 80 U/mL. Suitably, the collagenase type I may be provided in the cell culture at or below 50 U/mL. Suitably, the collagenase type I may be provided in the cell culture at or below 20 U/mL. Suitably, the collagenase type I may be provided in the cell culture at or below 10 U/mL. Suitably, the collagenase type I may be provided in the cell culture at or below 5 U/mL. Suitably, the collagenase type I may be provided in the cell culture at or below 3 U/mL.

Suitably, the collagenase type I may be provided in the cell culture at or below 2 U/mL.

Suitably, the collagenase type I may be provided in the cell culture at or below 1 U/mL or any range formed from any of the upper or lower limits of the aforementioned ranges, or any integer therebetween, or at least, no more than, below or above any value of an aforementioned ranges. In each of these cases the lower end of the range may be 0.005 U/mL e.g. 0.008 U/mL. For example, where the collagenase is collagenase type I, it may be provided in a cell culture at a concentration between 0.008 U/mL and 20 U/mL. For example, where the collagenase is collagenase type I, it may be provided in a cell culture at a concentration between 5 U/mL and 20 U/mL, or at a concentration between 9 U/mL and 20 U/mL. For example, where the collagenase is collagenase type I, it may be provided in a cell culture at a concentration between 0.008 U/mL and 10 U/mL. For example, where the collagenase is collagenase type I, it may be provided in a cell culture at a concentration between 5 U/mL and 10 U/mL, or at a concentration between 0.008 U/mL and 5 U/mL.

Suitably, where the collagenase is collagenase type VII, it may be provided in a cell culture at or below 500 U/mL. Suitably, the collagenase type VII is provided in a cell culture at or below 300 U/mL. Suitably, the collagenase type VII is provided in a cell culture at or below 200 U/mL. Suitably, the collagenase type VII is provided in a cell culture at or below 150 U/mL. Suitably, the collagenase type VII may be provided in a cell culture at or below 120 U/mL. Suitably, the collagenase type VII may be provided in the cell culture at or below 110 U/mL.

In each of these cases the lower end of the range may be 50 U/mL. For example, where the collagenase is collagenase type VII, it may be provided in a cell culture at a concentration between 50 U/mL and 300 U/mL. For example, where the collagenase is collagenase type VII, it may be provided in a cell culture at a concentration between 50 U/mL and 200 U/mL, or at a concentration between 50 U/mL and 150 U/mL. For example, where the collagenase is collagenase type VII, it may be provided in a cell culture at a concentration between 50 U/mL and 120 U/mL. For example, where the collagenase is collagenase type VII, it may be provided in a cell culture at a concentration between 50 U/mL and 110 U/mL.

Suitably, where the enzyme is dispase, it may be provided in the cell culture medium at or below 13 U/mL. Suitably, the dispase is present in the cell culture medium at or below 6.5 U/mL.

Suitably, the dispase is present in the cell culture medium at or below 3 U/mL. Suitably, the dispase is present in the cell culture medium at or below 2 U/mL or at or below 1 U/mL. Suitably, the dispase is present in the cell culture medium at or below 0.8 U/mL or at or below 0.5 U/mL. In each of these cases the lower end of the range may be 0.0005 U/mL e.g. 0.0008 U/mL. For example, the dispase may be provided in a cell culture at a concentration between 0.0008 U/mL and 3 U/mL. For example, the dispase may be provided in a cell culture at a concentration between 0.0008 U/mL and 2 U/mL, or at a concentration between 0.0008 U/mL and 1 U/mL. For example, the dispase may be provided in a cell culture at a concentration between 0.0008 U/mL and 0.8 U/mL. For example, the dispase may be provided in a cell culture at a concentration between 0.0008 U/mL and 0.5 U/mL.

An enzyme unit (U) is a measure of an enzyme's catalytic activity, and an enzyme unit (U) is the amount of enzyme which catalyses the conversion of one micromole of substrate per minute under the specified conditions. Alternatively, the enzyme activity may be expressed in katals (the enzyme activity which converts one mole of substrate per second under the specified conditions). For collagenase, the enzyme activity unit may be a Collagenase Degrading Units (CDU or Mandl) where one CDU catalyses the hydrolysis of one micromole of L-leucine equivalents from collagen in 5 hours at 37° C., pH 7.4 (Lockhardt et al J. Stem Cell Res Ther, 5:321 (2015)). The specified conditions according to the present invention are those defined in the Examples, for example a static or dynamic serum free culture, on glass microcarriers. An equivalent amount of enzyme units may be determined for different conditions or enzymes by the skilled person by measuring the enzyme activity under different conditions and adjusting the amount of enzyme to achieve the desired level of enzyme activity under the different conditions. For commercially available enzymes, the amount of enzyme as a measure of weight/volume (e.g. mg/mL) in the cell culture can be determined, for example by using the % enzyme and units of enzyme activity provided by the distributor. Thus for any commercially available enzyme, the skilled person can determine the amount of product required to provide the desired enzyme units. By way of example, for collagenase type I, the enzyme units of 110 U/mL is equivalent to 0.5 mg/ml based on the proportion of protease in the product, and the weight of product used in the cell culture. Similarly, the enzyme units of 215 U/mL for collagenase type I as used herein is equivalent to 1 mg/mL. For dispase I as used herein, enzyme units of 6.5 U/mL is equivalent to 0.5 mg/mL and enzyme units of 13 U/mL is equivalent to 1 mg/mL.

When expressed as a concentration of enzyme, an exogenous metalloprotease (e.g. collagenase and/or dispase) may be provided in the cell culture media at or below 1 mg/ml, or suitably at or below 0.5 mg/mL. Suitably, an exogenous protease (e.g. collagenase and/or dispase) may be provided at or below about 1 mg/mL, 0.9 mg/mL, 0.8 mg/mL, 0.7 mg/mL, 0.6 mg/mL, 0.5 mg/mL, 0.4 mg/mL, 0.3 mg/mL, 0.2 mg/mL, 0.1 mg/mL, more suitably at or below 0.09 mg/mL, 0.08 mg/mL, 0.07 mg/mL, 0.06 mg/mL, 0.05 mg/mL, 0.04 mg/mL, 0.03 mg/mL, 0.02 mg/mL, 0.01 mg/mL, 0.005 mg/mL, 0.001 mg/mL, or 0.0005 mg/mL. Suitably, the metalloprotease (e.g. collagenase and/or dispase) may be provided at a concentration in the cell culture media of about 0.0001 mg/ml to 1 mg/mL, 0.001 mg/mL to 0.9 mg/mL, 0.01 mg/mL to 0.9 mg/mL, 0.1 mg/mL to 0.9 mg/mL, or any range formed from any of the upper or lower limits of the aforementioned ranges, or any integer therebetween, or at least, no more than, below or above any value of an aforementioned ranges.

Suitably, the exogenous cysteine protease (e.g. papain and/or ficin) may be provided at or below about 1 mg/mL, 0.9 mg/mL, 0.8 mg/mL, 0.7 mg/mL, 0.6 mg/mL, 0.5 mg/mL, 0.4 mg/mL, 0.3 mg/mL, 0.2 mg/mL, 0.1 mg/mL, more suitably at or below 0.09 mg/mL, 0.08 mg/mL, 0.07 mg/mL, 0.06 mg/mL, 0.05 mg/mL, 0.04 mg/mL, 0.03 mg/mL, 0.02 mg/mL, 0.01 mg/mL, 0.005 mg/mL, 0.001 mg/mL, or 0.0005 mg/mL.

Suitably, ficin may be provided at a concentration in the cell culture media of about 0.0001 mg/ml to 1 mg/mL, 0.001 mg/mL to 0.75 mg/mL, 0.001 mg/mL to 0.5 mg/mL, 0.01 mg/mL to 0.25 mg/mL, or any range formed from any of the upper or lower limits of the aforementioned ranges, or any integer therebetween, or at least, no more than, below or above any value of an aforementioned ranges. In another example ficin may be provided at a concentration in the cell culture media of about from 0.005 mg/ml to 0.1 mg/mL, for example of about from 0.01 mg/mL to about 0.05 mg/mL.

Suitably, ficin may be provided at a concentration in the cell culture media of about from 0.0001 to about 0.1 BAPA U/mL, for example from about 0.0005 to about 0.01, from about 0.001 to about 0.009, or for example from about 0.002 to about 0.005 BAPA U/mL. Suitably, ficin may be provided at a concentration of about 0.0033 BAPA U/mL in the cell culture media. Suitably, papain may be provided at a concentration in the cell culture media of about 0.0001 mg/ml to 1 mg/mL, 0.001 mg/mL to 0.75 mg/mL, 0.001 mg/mL to 0.5 mg/mL, 0.005 mg/mL to 0.25 mg/mL, or any range formed from any of the upper or lower limits of the aforementioned ranges, or any integer therebetween, or at least, no more than, below or above any value of an aforementioned ranges. In another example papain may be provided at a concentration in the cell culture media of about from 0.0001 mg/ml to 0.07 mg/mL, for example of about from 0.001 mg/mL to about 0.025 mg/mL.

Suitably, papain may be provided at a concentration in the cell culture media of from about 0.001 to about 10 TU U/mL, for example from about 0.05 to about 5, from about 0.1 to about 2 or for example from about 0.5 to about 1 TU U/mL. Suitably, papain may be provided at a concentration of about 0.79 TU U/mL in the cell culture media.

The cells generated from the method of the invention may be used in a variety of research, diagnostic, drug screening, therapeutic, medical, industrial or food based applications. For example, a cell culture produced by a method of the present invention may find use in the cultured meat industry. Suitably, a method of the invention may comprise processing steps related to the application of the cells generated in the cell culture.

Throughout the description and claims of this specification, the words “comprise” and “contain” and variations of them mean “including but not limited to”, and they are not intended to (and do not) exclude other moieties, additives, components, integers, or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### EXAMPLES

### Example 1

Data have been generated using C2C12 as cell model grown in serum-free media (DMEM F12 with 1% Insulin Transferrin Factor and 1 mM of L-Ascorbic Acid).

1.1 Collagenase can Successfully Detach C2C12 Cells in Bulk without Altering their Adhesion, Proliferation, and Differentiation Potential.

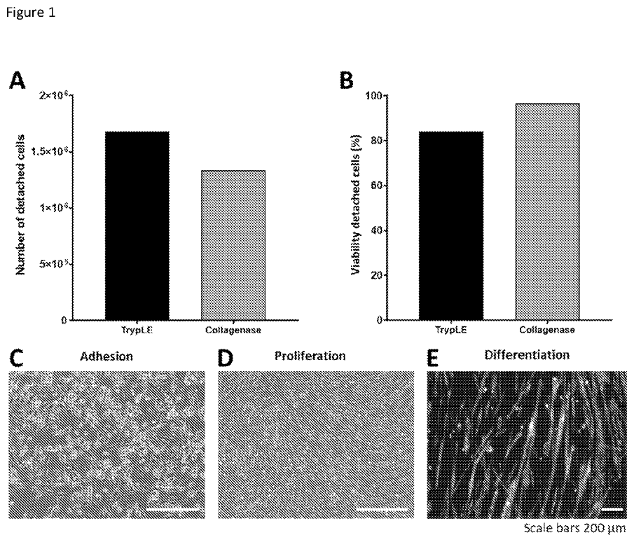

Collagenase has previously been used for the bulk detachment of some types of adherent cells in alternative to more common methods such as trypsin and TrypLE. In order to achieve bulk cell detachment in short periods of time a high concentration of the collagenase was added and incubated at 37° C. for 30 min (pre-warmed collagenase solution before addition). Collagenase type I concentration used: 1180 U (or 472 U/mL; which is equivalent to 2.2 mg/mL). Collagenase supplementation showed comparable results to TrypLE in terms of detachment efficiency (FIG. 1A) and viability (FIG. 1B) of the collected cells after detachment. Moreover, similarly to the control cells, the collagenase-detached cells can reattach, proliferate, and differentiate (FIG. 1C-E).

### 1.2. Collagenase is Capable of Detaching Cells in a Dose-Dependent Manner.

It was demonstrated that increasing amounts of collagenase lead to the detachment of an increasing number of C2C12 cells (Table 1).

TABLE 1
Quantification showing increasing number of detached cells using
Collagenase type I (units/cell) in a dose-dependent manner.

| | | | Detached |
|---|---|---|---|
| Collagenase | Collagenase | Collagenase | Number |
| [Units/cell] | [Units/mL] | [mg/mL] | of Cells |
| 3.92 × 10⁻⁵ | 2.625 | 0.012 | 445517 |
| 5.22 × 10⁻⁵ | 3.500 | 0.016 | 572663 |
| 6.53 × 10⁻⁵ | 4.375 | 0.020 | 594022 |

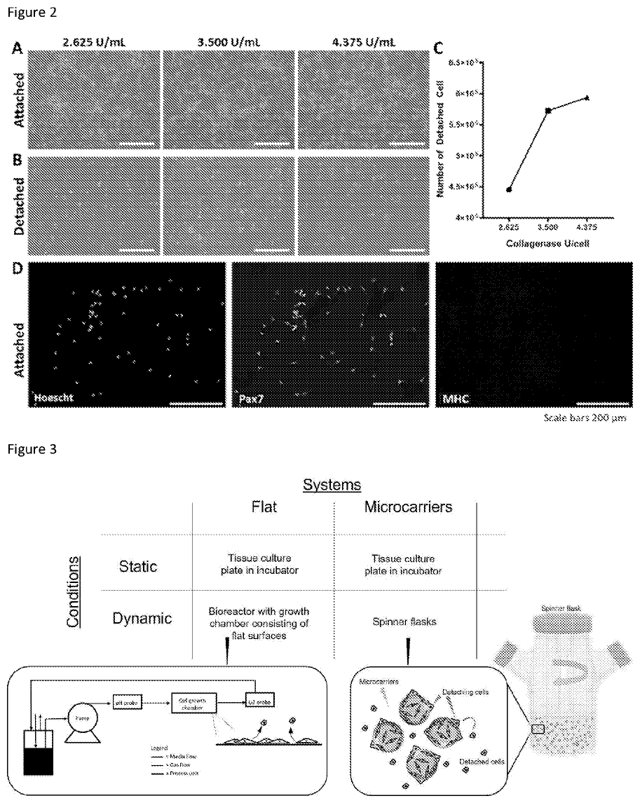

This suggested that collagenase could surprisingly be used to control the rate of cell detachment. Moreover, the detached cells were capable of reattaching and were not differentiated during the process (FIG. 2).

### 1.3. Low Concentrations of Collagenase Allow for Continuous Detachment of C2C12 Cells.

It was demonstrated that dosing the collagenase at lower concentrations allowed for continuous cell detachment without impacting cell characteristics. This was investigated in 4 different systems (see A to D below).

### A. Continuous Cell Detachment in a Flat System Under Static Conditions.

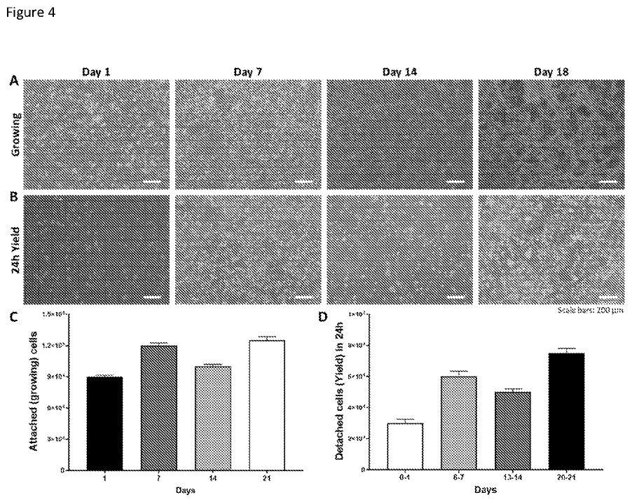

It was demonstrated that supplementing with 1.75 U/mL (equivalent to 0.0081 mg/ml of the Collagenase type I used herein) of collagenase type I allowed C2C21 cells to continuously grow and detach for 21 days in static culture in serum-free media (FIG. 4). Detached cells were collected every 24 hours and counted at day 1, 7, 9, 14 and 21. Over the time in culture, the confluence on the growing surface remained similar (FIG. 4A) without growing to over-confluency levels. Each surface with growing cells was moved to a different (new) well every 48 h and fresh media and collagenase was added. Cells detached from the surface at similar levels over the time (FIG. 4B).

### B. Continuous Detachment in a Flat System in Dynamic Conditions.

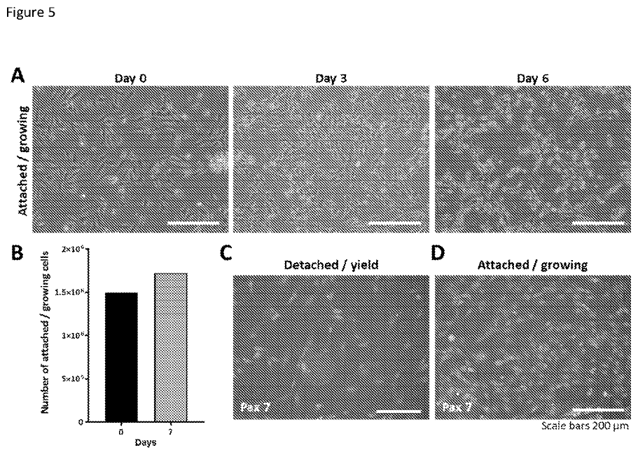

It was demonstrated that supplementing with 0.009 U/ml (equivalent to 0.00004 mg/mL (43.27 ng/ml) of the Collagenase type I used herein) of collagenase type I allowed C2C12 cells to continuously grow and detach for 7 days in dynamic culture in serum-free media (FIG. 5). Detached cells were collected at the end of the experiment whilst growing cells were imaged every day (FIG. 5A). At the end of the experiment, the number of harvested attached cells was similar to the number of seeded cells (FIG. 5B). In addition, both attached and detached cells displayed the initial undifferentiated phenotype at the end of the 7 days continuous culture under flow (FIGS. 5C&D).

### C. Continuous Detachment in Microcarrier System in Static Conditions.

It was demonstrated that supplementing with 4.5 U/mL of collagenase type I allowed C2C12 cells to continuously grow and detach for 27 days in static culture in serum-free media.

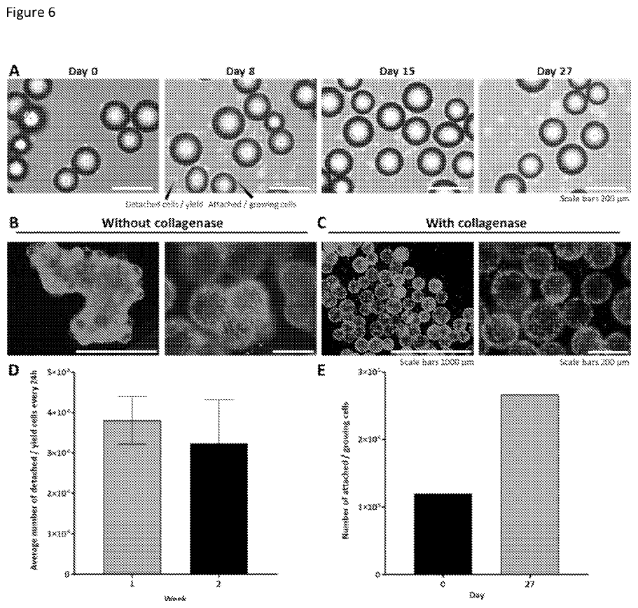

The confluence of the growing cells on the microcarriers was maintained during the time in culture and cell detachment was observed after supplementation of collagenase started at day 1 (FIG. 6A). The detached cells were collected every 24 h and the average number of cells that detached were consistent between the first and the second week (FIG. 6D), with around a third of the initially seeded cells detached every 24 h. The number of cells that were harvested from (previously growing on) the microcarriers did increase from day 1, however the cells did not overgrow on the microcarrier surface leading to aggregations (FIG. 6E).

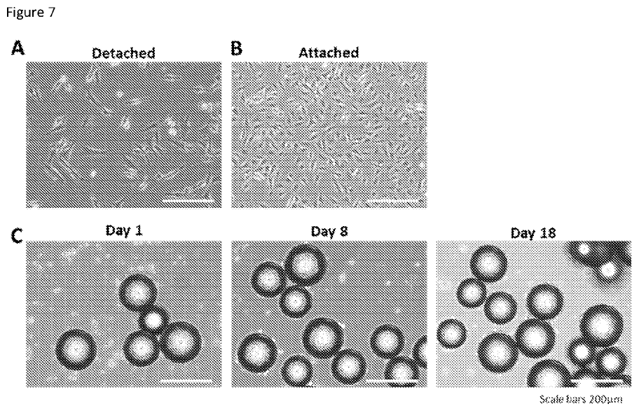

Moreover, both (i) the detached (yield) cells that detached during the process and (ii) the attached cells recovered from (growing on) the microcarriers at day 27, were capable of reattaching to the tissue culture plastic after collection and displayed the initial undifferentiated phenotype (FIGS. 7A&B). In addition, the detached (yield) cells were capable of re-attaching to fresh microcarriers in presence of collagenase and undergo the same process again (FIG. 7C).

### D. Continuous Detachment in Microcarriers System in Dynamic Condition.

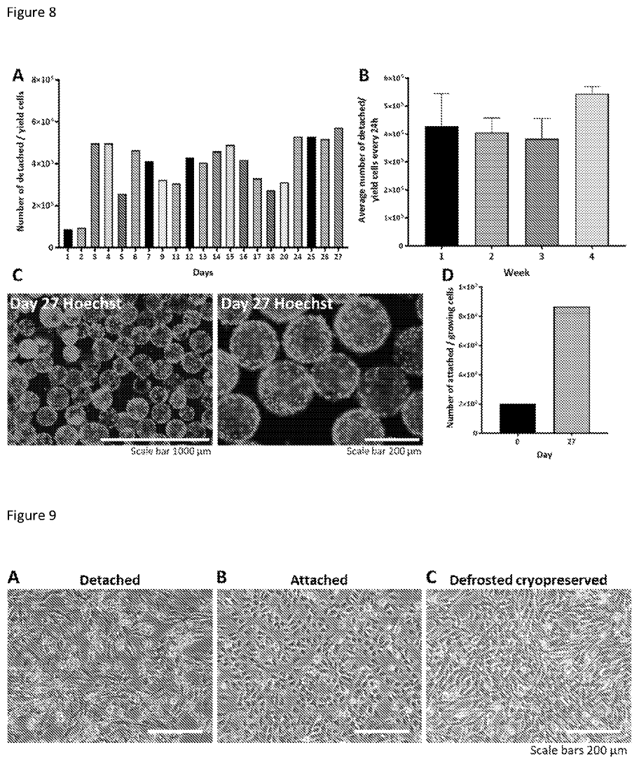

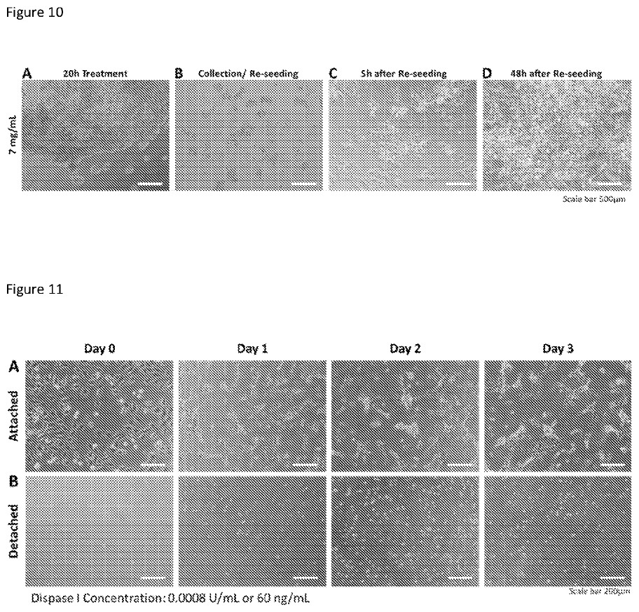

It was demonstrated that supplementing with 4.5 U/mL of collagenase type I allowed C2C12 cells to continuously grow and detach for 27 days in dynamic culture in serum-free media. Cell detachment was observed throughout the time in culture with collagenase supplementation (FIG. 8). Detached cells were collected every 24 h (FIG. 8A) and the average number of cells that detached has been consistent between the four weeks (FIG. 8B), with around a third of the initially seeded cells detaching every 24 h. The number of cells that were harvested from (previously growing on) the microcarriers did increase from day 1, however these cells did not overgrow on the microcarrier surface (FIG. 8D).

As observed in the static system, both (i) detached (yield) cells that detached during the process and (ii) attached cell recovered from (growing on) the microcarriers at day 27, were capable of reattaching to the tissue culture plastic after collection and displayed the initial undifferentiated phenotype (FIGS. 9A&B). Finally, the detached (yield) cells were capable of remaining functional after being cryopreserved for a few days (FIG. 9C).

### Dispase I

Different concentrations of dispase I were tested on C2C12 cells grown in serum-free culture media. Bulk detachment was achieved supplementing the media with 0.8 U/mL (equivalent to 0.062 mg/mL) of dispase I used herein. With serial dilutions starting from 0.8 U/mL (equivalent to 0.062 mg/mL) of dispase I, the inventors found that 0.0008 U/mL (equivalent to 0.00006 mg/mL (60 ng/ml)) allowed them to continuously detach C2C12 over a period of 5 days. This preliminary experiment suggests that dispase I could be used as an alternative to Collagenase type I (see FIG. 11).

### Collagenase VII (Pure Collagenase)

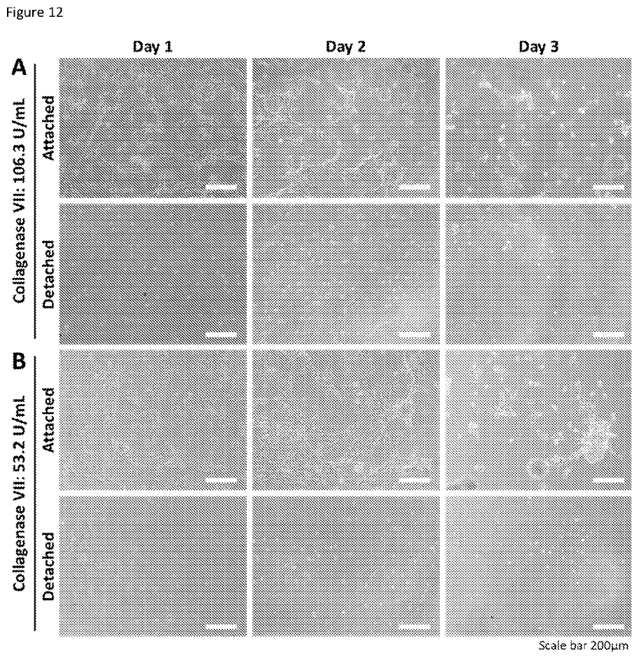

The inventors also tested pure collagenase (collagenase VII) to understand if it could also be used to achieve continuous cell detachment. A wide range of concentrations were tested, and continuous cell detachment was achieved using higher enzyme units compared to the crude reagent (collagenase type I). An optimal concentration for Collagenase VII was determined to be in the range between 106.35 U/mL and 53.18 U/mL [see FIG. 12]. Thus, pure collagenase is also able to continuously detach cells.

### Example 2

This example provides data supporting that cysteine proteases Ficin and Performase® can be used similarly to collagenase and dispase for continuous detachment of adherent cells and reducing aggregation of aggregation of microcarriers.

Data have been generated using C2C12 as cell model grown in serum-free media (DMEM F12 with 1% Insulin Transferrin Factor and 1 mM of L-Ascorbic Acid). The data provided below shows that Ficin and Performase® can:
i) detach cells in a dose-dependent manner;
ii) detach cells at steady-state without altering cell phenotype; and
iii) detach cells in bulk without compromising cell viability.

The two plant-based cysteine proteases that were tested are Ficin (product FSM200) and Performase® (product PSM100) purchased from Enzybel International S.A. Ficin and Papain (Performase® is derived from Papain) are cysteine proteases (they have a sulfhydryl group at their active site).

### Ficin

Ficin (historically called Ficain) was purified from the latex of fig tree Ficus glabrata or Ficus anthelmintica and is part of Cysteine endopeptidase family. This Ficin is a food-grade natural protease. Optimum working conditions are pH 5-9 and Temp 40-75° C. The CoA (Certificate of Analysis) from Enzybel stated the active units of this batch as 213 BAPA/g (*). Such enzyme is already used for food production and pharmaceuticals applications.

### Performase®

Performase® is an enzyme preparation derived from papaya (peptidase-papain); it is food grade and containing endopeptidase. The CoA from Enzybel stated active units of this batch as 103 TU/mg (**). Performase® is already used for food & beverages, and pharmaceuticals applications. Origin: Garica papaya L.

### *Principle of the BAPA Method:

For the analysis of enzymatic (proteolytic) activity of Ficin, a synthetic substrate is used N-α-benzoyl-DL-arginine-p-nitroanilide hydrochloride (DL-BAP (N) A), which is cleaved by the enzyme. The substance “p-nitroanilin” (cleaved fragment) gives an indication on the hydrolysis rate during the reaction. This substance is measured photometrically. One unit of Ficin activity corresponds with the quantity of enzyme that hydrolyses the equivalent of 1 μmol of the substrate per minute under the conditions of the assay.

### **Principle of the TU Method:

Enzyme solution added to a buffered solution of casein which is digested at 40° C. and pH 6.0 for 60 minutes. The reaction is stopped, and the excess of casein is precipitated with trichloracetic acid. The digest is filtered, and the optical density of the clear filtrate is read at 280 nm. The activity of the enzyme is then calculated from the observed optical density and the slope of a Standard Tyrosine curve. TU unit (TU/mg) is defined as the unit which, while acting on a casein substrate under specific conditions, releases 1 μg of Tyrosine per minute.

### 2.1 Dose-Dependent Cell Detachment

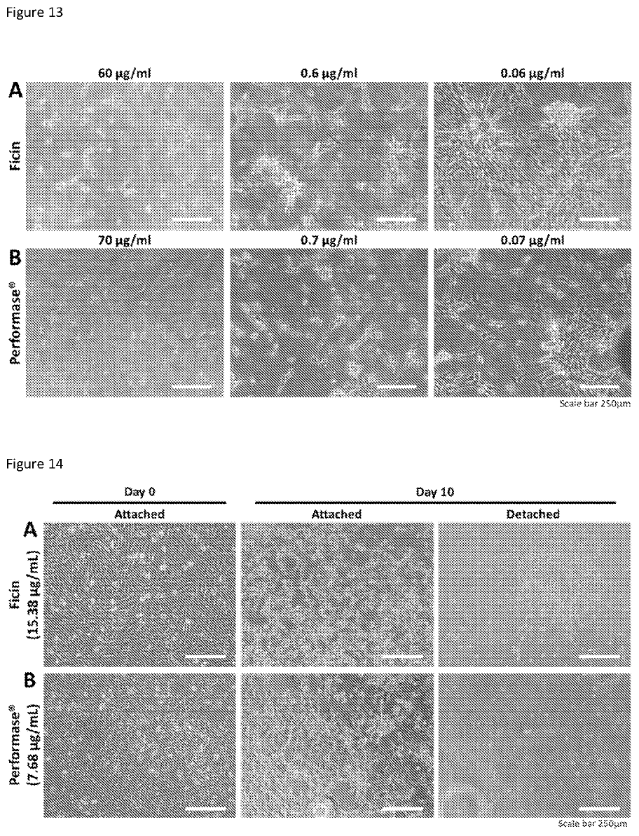

Similarly to collagenase and dispase, both Ficin and Performase® are capable of detaching cells in a dose-dependent manner. Hence, increasing concentrations of enzyme detach an increased number of cells from the surface. FIG. 13 shows images of cells growing on surfaces after 6 days of incubation with different enzyme concentrations. The concentrations of Ficin (FIG. 13A) used were 0.06 μg/mL, 0.6 μg/mL, and 60 μg/mL. The concentrations of Performase® (FIG. 13B) used were 0.07 μg/mL, 0.7 μg/mL, and 70 μg/mL. As the enzyme's concentrations increased, the number of detached cells increased (numerical data not shown, but observable in FIG. 13) leaving less cells growing on the original surface. Hence, there is a dose-dependency between the enzyme concentration and the number of detached cells. This was confirmed for both enzymes (FIG. 13)

### 2.2 Continuous Cell Detachment

Similarly to collagenase and dispase, both Ficin and Performase® are capable of detaching cells at a steady-state. Gathered data shows continuous growth and detachment of C2C12 cells over 10 days in a static system in serum-free media (FIG. 14)

Images in FIG. 14 shows (A) Ficin at 15.36 μg/mL (0.0033 BAPA U/mL) and (B) Performase® at 7.68 μg/mL (0.79 TU U/mL) were capable to maintain over 10 days a similar confluency on the proliferating surface (attached) whilst cells were continuously detaching from it (detached).

Moreover, immunostaining shows that the process did not interfere with the phenotype of both attached and detached cells. Indeed, Pax7 positivity was found above 90% in all conditions (data not shown).

### 2.3 Bulk Cell Detachment

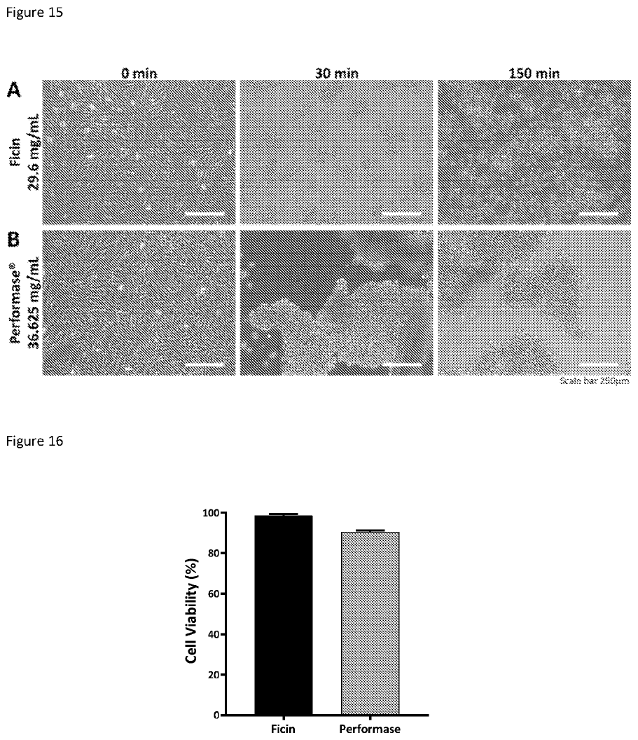

Similarly to collagenase and dispase, both Ficin and Performase® can detach cells in bulk once added to the cell culture media. In this experiment C2C12 cells were seeded at a density of 50,000/cm2 in a 24 well plate with 1 mL of serum free media (SFM). Cells were allowed to adhere to the surface overnight. Each enzyme was then prepared in SFM and 1 mL added to each well at concentrations ranging from 29.6 mg/mL (6.3 BAPA U/mL, FIG. 15A) to 1.85 mg/mL (0.39 BAPA U/mL) for ficin and between 36.625 mg/mL (3772 TU U/mL, FIG. 15B) to 2.29 mg/ml (236 TU U/mL) for Performase®. Both enzymes at the tested concentrations could efficiently detach cells from the surface already 30 minutes after their addition (FIG. 15). Complete detachment occurred after 150 minutes (FIG. 15) for both enzymes.

### Cell Viability

Moreover, the addition of Ficin and Performase® did not compromise cell viability during the total 4 h of incubation with the enzymes. The detached cells were indeed collected, centrifuged, and resuspended in serum-free media (SFM) for cell count & viability. Viability of such detached cells was found to be 90.5% and 98.5% when detached with Performase® and ficin, respectively (FIG. 16).

### CONCLUSION

Ficin and Performase® are capable of continually detaching adherent cells at a steady-state without altering cell viability and phenotype when added at low concentrations to the culture media.

1. A method of reducing aggregation in a cell cluster-based cell culture system, the method comprising the step of:
(i) culturing a cell population comprising two or more cell clusters in a cell culture medium for a culture period, wherein the cell culture medium comprises an exogenous protease, wherein the exogenous protease is an exogenous metalloprotease and/or cysteine protease.

2. The method of claim 1, wherein the two or more cell clusters each comprise a microcarrier.

3. The method of claim 1, wherein each of the two or more cell clusters is a spheroid, an organoid or a tissue.

4. The method of any one of the preceding claims, wherein the aggregation is cell-mediated aggregation.

5. The method of any one of the preceding claims, wherein the culture period of (i) comprises all or part of the log phase of growth of the cell population.

6. The method of any one of the preceding claims, wherein the culture period is at least 24 hours.

7. The method of any one of the preceding claims, wherein the cell culture system is a continuous cell culture system or a batch cell culture system, optionally wherein the system comprises a bioreactor.

8. A cell culture vessel comprising a cell culture medium, two or more cell clusters and an exogenous protease, wherein the two or more cell clusters comprise a cell population in the log phase of growth and wherein the exogenous protease is an exogenous metalloprotease and/or cysteine protease.

9. The cell culture vessel of claim 8, wherein the vessel is part of a continuous cell culture system or a batch cell culture system, optionally wherein the system comprises a bioreactor.

10. The method of any one of claims 1 to 7, or the cell culture vessel of claim 8 or 9, wherein the metalloprotease is collagenase and/or dispase.

11. The method or the cell culture vessel of claim 10, wherein the collagenase is collagenase type I or collagenase type VII.

12. The method or the cell culture vessel of claim 10, wherein the dispase is dispase I.

13. The method of any one of claims 1 to 7, or the cell culture vessel of claim 8 or 9, wherein the cysteine protease is of the CA clan.

14. The method or the cell culture vessel of claim 13, wherein the cysteine protease of the CA clan is be selected from the group consisting of ficin, papain, bromelain, cathepsin K and calpain.

15. The method or cell culture vessel of any one of the preceding claims, wherein the two or more cell clusters each comprise a microcarrier, and the microcarrier is a bead.

16. The method or cell culture vessel of any one of the preceding claims, wherein the two or more cell clusters each comprise a microcarrier and wherein the microcarrier comprises a material selected from the group consisting of: plastic, polymer, glass, and metal.

17. The method or cell culture vessel of any one of the preceding claims, wherein the cell population comprises cells selected from the group consisting of: skin, muscle, cervical, breast, and prostate cells.

18. The method of any one of claims 1 to 7 or 10 to 17, wherein the cell culture is supplemented with the protease one or more times during the step of culturing.

19. The method of any one of claims 1 to 7 or 10 to 18, wherein the method comprises the step of culturing the cell population in a cell culture medium lacking the protease before and/or after step (i).

20. The method of any one of claims 1 to 7 or 10 to 19, wherein the protease is present in the cell culture medium at a concentration which maintains the cell culture at about 50% to 100% confluency.

21. The method of any one of claims 1 to 7 or 10 to 20, wherein the culturing step does not comprise a step of washing the cells.

22. The method of any one of claims 1 to 7 or 10 to 21, wherein the protease is present in the cell culture medium at a concentration which allows for continuous detachment of a portion of the cells from the cell clusters.

23. The method of claim 22, wherein the protease is present in the cell culture medium at a concentration which allows for the ratio of the rate of detachment of cells to the rate of growth of the cells to be in the range of 0.3:1 to 1:3.

24. The method of any one of claims 22 to 23, wherein the detached cells, or a proportion of the detached cells, are harvested at least once during the culturing step.

25. The method of any one of claims 22 to 24, wherein the method comprises adjusting the protease concentration and/or frequency of application to adjust the rate of cell detachment, wherein increasing the concentration and/or frequency of application increases the rate of continuous cell detachment, and decreasing the concentration and/or frequency of application decreases the rate of continuous cell detachment.
